(19) United States
(12) Patent Application Publication
(10) Pub. No.: US 2019/0270689 A1
(43) Pub. Date: Sep. 5, 2019

(54) Radiolabeling Agents, Methods of Making, and Methods of Use Thereof

(71) Applicant: The United States of America, as Represented by The Secretary, Department of Health and Human Servi, Bethesda, MD (US)

(72) Inventors: Victor William PIKE, Bethesda, MD (US); Mohammad Baqir HASKALI, Bethesda, MD (US); Bo Yeun YANG, Bethesda, MD (US); Sanjay TELU, Bethesda, MD (US)

(21) Appl. No.: 16/349,124

(22) PCT Filed: Nov. 9, 2017
(86) PCT No.: PCT/US2017/060838

§ 371 Date: May 10, 2019

Related U.S. Application Data
(60) Provisional application No. 62/420,840, filed on Nov. 11, 2016.

(51) Int. Cl. C07C 19/14; C07F 1/08; C07B 59/00

(52) U.S. Cl. CPC C07C 19/14; C07F 1/08; C07C 17/10; C07B 2200/05; C07B 59/001

(57) ABSTRACT

Described herein are labeling agents, specifically [¹¹C]fluoroform, [¹¹C]difluoromethane, [¹¹C]fluoromethyl iodide, [¹¹C]fluoromethyl bromide, [¹¹C]fluoromethyl chloride, [¹¹C]fluoromethyl trifluoromethansulfonate, [¹¹C]difluoromethyl iodide, [¹¹C]difluoromethyl bromide, [¹¹C]difluoromethyl chloride, [¹¹C]difluoromethyl trifluoromethansulfonate, [¹¹C]trifluoromethyl iodide, [¹¹C]trifluoromethyl bromide, [¹¹C]trifluoromethyl chloride, [¹¹C]trifluoromethyl trifluoromethansulfonate, [¹⁸F]fluoroform, [¹⁸F]difluoromethane, [¹⁸F]difluoromethyl bromide or [¹⁸F]trifluoromethyl bromide. Also included are methods of labeling precursors to provide labeled fluoroalkanes and imaging methods.

### FIELD OF THE DISCLOSURE

The present disclosure pertains to radiolabeling agents, methods of making the radiolabeling agents and labeled molecules at high molar activity and their use in imaging methods such as positron emission tomography (PET).

### BACKGROUND

Positron emission tomography (PET) is increasingly important for biomedical research, and for drug discovery and development. The value of PET for imaging a particular molecular target in a living human or animal subject depends on a biochemically specific radiotracer being available, where the radiolabel is usually one of the short-lived positron-emitters, carbon-11 (t_{1/2}=20.4 min) or fluorine-18 (t_{1/2}=110 min). Because of the short half-lives of these radionuclides, a derived ¹¹C-labeled radiotracer must generally be produced within minutes of use and a derived ¹⁸F-labeled radiotracer within hours of use. PET radiotracer production methods must therefore be repetitively reliable.

The molecular position of the radiolabel is often critical for the efficacy of the radiotracer, and in particular for avoiding troublesome radiometabolites that may confound attempts to quantify radiotracer interaction with the imaging target. Thus, in some cases, it may be preferable to have a carbon-11 label and in others a fluorine-18 label, or to label in one part of the structure rather than in another.

A further consideration is the molar activity of the radiotracer, namely the ratio of its radioactivity (Bq) to the mass of tracer (mol), where the latter is predominantly the accompanying non-radioactive tracer (which is commonly known as carrier). For imaging targets that exist at only low density in vivo, such as many enzymes, transporters, receptors and plaques, the radiotracer molar activity needs to be as high as possible. A low molar activity (corresponding to a high amount of carrier) may result in high occupancy of the target binding site by carrier with consequent violation of the tracer principle, or even to an obliteration of any target-specific signal. Minimal occupancy of the target by carrier may also aid in preventing unwanted pharmacological effects. High molar activity (corresponding to a low amount of carrier), i.e., exceeding 40 GBq/μmol, is generally desirable for PET radiotracers intended to image low density protein targets in vivo.

Over recent decades, the most popular methods for labeling PET radiotracers at high molar activity (>50 GBq/μmol) have used [¹¹C]methyl iodide or [¹⁸F]fluoride ion as labeling agents. [¹¹C]Methyl iodide is produced rapidly and efficiently from cyclotron-produced [¹¹C]methane or [¹¹C]carbon dioxide, whereas [¹⁸F]fluoride ion is produced directly from a cyclotron. [¹¹C]Methane and [¹¹C]carbon dioxide may be produced in very high activities (>100 GBq) from modern biomedical cyclotrons. [¹⁸F]Fluoride ion may be produced from a biomedical cyclotron in exceptionally high activities (approximately 1 TBq). These high activities are in strikingly high contrast with the relatively low activity of radiotracer that might be required for administration in a single PET examination (typically, approximately 75 MBq).

However, the use of [¹¹C]methyl iodide or [¹⁸F]fluoride ion and of other labeling agents, such as [¹¹C]methyl triflate (itself derived from [¹¹C]methyl iodide), restricts the kind of groups that might be labeled in radiotracers, for example to methyl (Me) groups for [¹¹C]methyl iodide or [¹¹C]methyl triflate, and to monofluoro (C-F) groups for [¹⁸F]fluoride ion.

Many small molecule drugs and potential PET radiotracers carry trifluoromethyl (CF₃) groups. In many cases, a hydrogen, methyl, fluoro, chloro or other substituent can be replaced by a CF₃ group with retention of similar physicochemical and pharmacological properties, or even potentially in some other cases with beneficial improvement in these properties. Also, generally, the CF₃ group is considered to be metabolically stable. These factors have led to strong interest from pharmaceutical companies in developing drugs that have CF₃ groups. In parallel, academic groups have expended considerable efforts on developing methods for labeling such groups with fluorine-18, with the most recent methods being based on conversion of cyclotron-produced [¹⁸F]fluoride ion into [¹⁸F]fluoroform (where fluoroform is CHF₃), and then in-situ generation of the reactive derivative [¹⁸F]CuCF₃. Because [¹⁸F]fluoroform and [¹⁸F]CuCF₃ have been produced by chemistry in solution, their potential utilities are constrained; moreover, if these reagents are not isolated before use, the necessary rapid purifications of labeled products can be challenging.

To date, methods for producing [¹⁸F]fluoroform and [¹⁸F]CuCF₃ have delivered at best only moderate molar activity (<32 GBq/μmol) due to inevitable [¹⁸F]fluoride ion dilution with the carrier fluoride ion that is generated in the reaction systems. In general, this is because the radioactive fluorine atom is the last to be introduced into the trifluoromethyl group, and the two earlier introduced non-radioactive fluorine atoms are therefore available for exchange. As stated above, the molar activities needed for radiotracers to be used for PET imaging of low density protein targets are generally much higher. The range of useful PET radiotracers that may be produced from [¹⁸F]fluoroform or [¹⁸F]CuCF₃ that is produced by the known best-performing methods is therefore limited to those not requiring such high molar activities.

What is needed are new labeling agents for PET radiotracers, new methods of making labeling agents with high molar activity, and new methods of making radiotracers at high molar activity.

### BRIEF SUMMARY

In an aspect, a labeling agent is [¹¹C]fluoroform, [¹¹C]difluoromethane, [¹¹C]fluoromethyl iodide, [¹¹C]fluoromethyl bromide, [¹¹C]fluoromethyl chloride, [¹¹C]fluoromethyl trifluoromethansulfonate, [¹¹C]difluoromethyl iodide, [¹¹C]difluoromethyl bromide, [¹¹C]difluoromethyl chloride, [¹¹C]difluoromethyl trifluoromethansulfonate, [¹¹C]trifluoromethyl iodide, [¹¹C]trifluoromethyl bromide, [¹¹C]trifluoromethyl chloride, or [¹¹C]trifluoromethyl trifluoromethansulfonate.

In another aspect, a labeling agent is [¹⁸F]fluoroform, [¹⁸F]difluoromethane, [¹⁸F]difluoromethyl bromide, or [¹⁸F]trifluoromethyl bromide.

In another aspect, a gas phase solvent-free method for producing a ¹¹C- or ¹⁸F-labeled fluoroalkane comprises

contacting [¹¹C]methane, [¹⁸F]fluoromethane, [¹¹C]methyl iodide, [¹¹C]methyl bromide, [¹¹C]methyl chloride, [¹¹C]methyl trifluoromethansulfonate, or [¹⁸F]fluoromethyl bromide with CoF₃ at a temperature from 50 to 450° C., and

isolating the ¹¹C- or ¹⁸F-labeled fluoroalkane that is produced.

In another aspect, a method of preparing a ¹¹C-labeled or ¹⁸F-labeled radiotracer comprises combining [¹¹C]fluoroform or [¹⁸F]fluoroform with a non-radioactive precursor to form a reaction mixture, and producing the ¹¹C-labeled radiotracer or the ¹⁸F-labeled radiotracer from the reaction mixture, wherein the non-radioactive precursor contains a functionality that is reactive with the [¹¹C]fluoroform or the [¹⁸F]fluoroform.

In another aspect, a method of preparing an ¹¹C-labeled or ¹⁸F-labeled radiotracer comprises converting [¹¹C]fluoroform or [¹⁸F]fluoroform into [¹¹C]CuCF₃ or [¹⁸F]CuCF₃, combining the [¹¹C]CuCF₃ or [¹⁸F]CuCF₃ with a non-radioactive precursor to form a reaction mixture, and producing the ¹¹C-labeled radiotracer or the ¹⁸F-labeled radiotracer from the reaction mixture, wherein the non-radioactive precursor contains a functionality that is reactive with the [¹¹C]CuCF₃ or [¹⁸F]CuCF₃.

In yet another aspect, an apparatus for gas phase production of a radiolabeled [¹¹C]- or [¹⁸F]-fluoroalkane from a ¹¹C- or ¹⁸F-labeled precursor comprises an inlet for radiolabeled precursor in fluid communication with an impurity trap for removal of impurities from the radiolabeled precursor, the impurity trap in fluid communication with a column for removal of ammonia and/or water from the radiolabeled precursor, the column in fluid communication with a furnace suitable for heating CoF₃ to a temperature of 50 to 450° C., the furnace optionally in fluid communication with a hydrogen fluoride trap, and the furnace or the hydrogen fluoride trap in fluid communication with a product trap.

### BRIEF DESCRIPTION OF THE DRAWINGS

FIG. 1 is a schematic of an apparatus for producing no-carrier-added (NCA) [¹¹C]fluoroform from cyclotron-produced [¹¹C]methane. Cyclotron-produced [¹¹C]methane in nitrogen-10% hydrogen is first passed though a stainless steel U-tube cooled in liquid argon (−186° C.) to trap out any impurities and then into a second U-tube containing PORAPAK™ Q cooled in liquid argon to trap the [¹¹C]methane. Waste gas goes to a collection bag. The U-tube is then raised and allowed to warm to room temperature (RT) over 4 min while the tube is flushed with helium gas (20 mL/min) to transfer the [¹¹C]methane over Sicapent® (for removal of any water or ammonia) and then into a stainless steel tube containing cobalt(III) fluoride (CoF₃; 17-19 g) heated at 270° C. The effluent is passed through acetonitrile-dry-ice (approximately −41° C.) to trap any trace hydrogen fluoride (b.p. 19.5° C.) and then into a trap containing ethanol that is cooled with hexane/liquid nitrogen (approximately −94° C.) or DMF cooled with acetonitrile-dry ice (approximately −41° C.) in order to trap the generated [¹¹C]fluoroform for subsequent reactions. The assembled apparatus is purged with helium at RT before use, and also left filled with helium between uses.

FIG. 2 shows a radio-HPLC analysis of [¹¹C]fluoroform that has been trapped in cold ethanol solution.

FIG. 3 shows the dependence of yield of [¹¹C]fluoroform from [¹¹C]methane on CoF₃ temperature. Values are means±S.D (n>3).

FIG. 4 shows the dependence of [¹¹C]fluoroform yield from [¹¹C]methane with the number of consecutive column uses for [¹¹C]fluoroform synthesis. Data shown are for the column operating at 270° C. with helium flow set at 20 mL/min, and with labeled products trapped in cold ethanol.

FIG. 5 is a schematic of the preparation and use of [¹¹C]fluoroform for labeling model organic compounds in trifluoromethyl groups. [¹¹C]Fluoroform may react directly with ketones or disulfides, in particular diaryl ketones or diaryl disulfides, as in the top examples, or may be rapidly converted into Cu¹¹CF₃ for reaction with, for example, arylboronic acids, aryl iodides, arenediazonium salts, aryl (vinyl)iodonium salts, or diaryliodonium salts, as shown in the bottom examples. The generally high speeds and efficiencies of these reactions under mild conditions are highly attractive for applications to the labeling of PET radiotracers with short-lived carbon-11.

FIG. 6 shows the radiochromatogram from the analysis of the reaction product from the reaction of [¹¹C]fluoroform with benzophenone.

FIG. 7 shows a reaction scheme and HPLC chromatogram of the crude [¹¹C]1-nitro-4-(trifluoromethyl)benzene (retention time approximately 5.5 min) from the reaction of 4-nitrobenzenediazonium tetrafluoroborate with [¹¹C]CuCF₃. The bottom curve represents radioactivity. The top curve represents UV absorbance.

FIG. 8 shows examples of ¹¹C-labeled drugs prepared from NCA [¹¹C]fluoroform.

FIG. 9 is a schematic of an apparatus for producing [¹⁸F]fluoroform from cyclotron-produced NCA [¹⁸F]fluoride ion via conversion of the [¹⁸F]fluoride ion into [¹⁸F]fluoromethane by reaction with methyl mesylate in DMSO in the presence of K⁺-K 2.2.2 complex in a Tracerlab Fx_{FN} apparatus. At the end of the reaction, the solvent in the reactor is purged with nitrogen to release the generated [¹⁸F]fluoromethane through a trap cooled in dry-ice to remove any trace solvent and then into a U-tube containing PORAPAK™ Q cooled in liquid argon, which traps the [¹⁸F]fluoromethane. Waste gas goes to a collection bag. The U-tube is then raised and allowed to warm RT over 4 min while the tube is flushed with helium gas (20 mL/min) to transfer the [¹⁸F]fluoromethane over Sicapent® (for removal of any water) and then into a stainless steel tube containing CoF₃ (17-19 g) heated at 280° C. The effluent is passed through acetonitrile-dry-ice (approximately −41° C.) to trap any trace hydrogen fluoride (b.p. 19.5° C.) and then into a cold trap containing ethanol or DMF cooled with hexane/liquid nitrogen (approximately −94° C.), or DMF cooled with acetonitrile-dry ice (approximately −41° C.) in order to trap the generated [¹⁸F]fluoroform for subsequent reactions. The assembled apparatus is purged with helium at RT before use, and also left filled with helium between uses.

FIG. 10 shows dependence of recovery yield of [¹⁸F]fluoromethane from [¹⁸F]fluoride ion on methylation reaction temperature.

FIG. 11 shows dependence of yield of [¹⁸F]fluoroform from [¹⁸F]fluoromethane on CoF₃ temperature and amount

FIG. 12 shows dependence of recovery yield and purity of [¹⁸F]fluoroform from [¹⁸F]fluoromethane on CoF₃ temperature.

FIG. 13 shows yield of [¹⁸F]fluoroform from [¹⁸F]fluoroform recovery yield and purity.

FIG. 14 shows a radio-HPLC analysis of [¹⁸F]fluoroform that has been trapped in cold ethanol solution.

FIG. 15 shows the yield of [¹⁸F]fluoroform from [¹⁸F]fluoromethane with respect to number of consecutive CoF₃ column uses.

FIG. 16 shows the molar activity changes in single CoF₃ column. The closed circles are the molar activity (ASm, GBq/μmol) and the closed squares are the molar activity are the [¹⁸F]fluoroform recovery yield in %.

FIG. 17 summarizes radiochemistry performed with [¹⁸F]fluoroform or the derivative [¹⁸F]CuCF₃.

FIG. 18 shows the radiochromatogram from the analysis of the reaction product from the reaction of [¹⁸F]fluoroform with benzophenone.

The above-described and other features will be appreciated and understood by those skilled in the art from the following detailed description, drawings, and appended claims.

### DETAILED DESCRIPTION

Described herein are novel labeling agents for PET radiotracers, methods of making labeling agents with high molar activity, and methods of labeling radiotracers, also in high molar activity. Exemplary labeling agents include [¹¹C]fluoroform and [¹⁸F]fluoroform, and the respective derivatives [¹¹C]CuCF₃ and [¹⁸F]CuCF₃.

The preparations of [¹¹C]fluoroform and [¹¹C]CuCF₃ have not been reported previously.

Labeling of the trifluoromethyl group with carbon-11 has never been achieved at any molar activity in any other reported work.

[¹¹C]Methane can be produced by the nuclear reaction ¹⁴N(p,α)¹¹C in very high molar activity that is at least comparable to the molar activities of other cyclotron sources of carbon-11 and fluorine-18. [¹¹C]Methane can also be produced efficiently and rapidly from cyclotron-produced [¹¹C]carbon dioxide by a rapid single pass over a heated nickel catalyst without any appreciable addition of carrier methane. These production methods are widely implemented in numerous PET research facilities.

The present inventors reasoned that if [¹¹C]fluoroform could be produced from readily accessible [¹¹C]methane, very high molar activity should be retained, and should be expected to exceed that currently achievable for [¹⁸F]fluoroform or [¹⁸F]CuCF₃.

The present inventors also reasoned that direct generation of [¹¹C]fluoroform in the gas phase in the absence of reagents or solvents would be attractive for broad unrestricted utility in labeling reactions.

The present inventors also reasoned that the previously unknown derivative [¹¹C]CuCF₃ might be prepared both rapidly and efficiently.

The inventors anticipated that convenient access to [¹¹C]fluoroform and the derivative [¹¹C]CuCF₃ would greatly expand the chemical space that is open for developing new PET radiotracers at high molar activity.

The inventors also considered that [¹¹C]fluoroform and its derivative [¹¹C]CuCF₃ could be applied in labeling methods without further dilution of molar activity, whereas several known methods of labeling with [¹⁸F]fluoroform or the derivative [¹⁸F]CuCF₃ risk dilution of molar activity because of the presence of fluorine-containing adjuncts in the reaction systems. For example, [¹⁸F]CuCF₃ is usually ‘stabilized’ in reaction media with trimethylamine trihydrofluoride or triethylamine trihydrofluoride (Et₃N.3HF).

In addition to producing [¹¹C]fluoroform, the methods disclosed herein can be used to produce other [¹¹C]fluoroalkanes, such as [¹¹C]difluoromethane (from [¹¹C]methane), [¹¹C]fluoromethyl iodide (from [¹¹C]methyl iodide), [¹¹C]fluoromethyl bromide (from [¹¹C]methyl bromide), [¹¹C]fluoromethyl chloride (from [¹¹C]methyl chloride) [¹¹C]fluoromethyl trifluoromethansulfonate (from [¹¹C]methyl trifluoromethansulfonate), [¹¹C]difluoromethyl iodide (from [¹¹C]methyl iodide), [¹¹C]difluoromethyl bromide (from [¹¹C]methyl bromide), [¹¹C]difluoromethyl chloride (from [¹¹C]methyl chloride) [¹¹C]difluoromethyl trifluoromethansulfonate (from [¹¹C]methyl trifluoromethansulfonate), [¹¹C]trifluoromethyl iodide (from [¹¹C]methyl iodide), [¹¹C]trifluoromethyl bromide (from [¹¹C]methyl bromide), [¹¹C]trifluoromethyl chloride (from [¹¹C]methyl chloride), or [¹¹C]trifluoromethyl trifluoromethansulfonate (from [¹¹C]methyl trifluoromethansulfonate).

The methods can also be used to provide an alternative synthesis of [¹⁸F]fluoroalkanes, such as [¹⁸F]fluoroform (from [¹⁸F]fluoromethane) and [¹⁸F]difluoromethane (from [¹⁸F]fluoromethane).

In addition, [¹⁸F]difluoromethyl bromide and [¹⁸F]trifluoromethyl bromide can be made from the precursor [¹⁸F]fluoromethyl bromide.

As used herein, the type of fluorination apparatus described for the production of [¹¹C]fluoroalkanes can also be used to produce [¹⁸F]fluoroalkanes.

Also labeling methods performed with [¹¹C]fluoroalkanes can also be performed with [¹⁸F]fluoroalkanes (and vice versa).

It will be appreciated that the described methods can also be used to prepare ¹³C enriched products, simply by using ¹³C-enriched material in place of the ¹¹C-labeled material (the isotopologue).

In an aspect, the ¹¹C-labeling agents described herein are no-carrier-added (NCA). As used herein, NCA means that the radiochemical processing of a starting cyclotron-produced material, such as [¹¹C]methane, does not intentionally add appreciable amounts of the non-radioactive species (carrier) at any stage of the process. Consequently, the high molar radioactivity of the cyclotron-produced starting material is well maintained throughout the process, when correction is made for radioactive decay. For example, NCA [¹¹C]fluoroform has a high ratio of [¹¹C]fluoroform (measured in Bq) to all other non-radioactive forms of fluoroform (measured in micromoles), which allows for the labeling of PET radiotracers at adequately high molar activity for imaging low density proteins in vivo with PET, or for any other purpose. Generally, NCA ¹¹C-labeling agents have molar activities exceeding 35 GBq/μmol. Much higher molar activities are possible, depending on their production parameters (e.g., such as the total amount of radioactivity produced from a particular cyclotron irradiation). In a specific aspect, the labeling agent is NCA and with a molar activity greater than 200 GBq/μmol, wherein the molar activity is corrected to the end of radionuclide production.

In an aspect, described herein, is a labeling agent that is [¹¹C]fluoroform, [¹¹C]difluoromethane, [¹¹C]fluoromethyl iodide, [¹¹C]fluoromethyl bromide, [¹¹C]fluoromethyl chloride, [¹¹C]fluoromethyl trifluoromethansulfonate, [¹¹C]difluoromethyl iodide, [¹¹C]difluoromethyl bromide, [¹¹C]difluoromethyl chloride [¹¹C]difluoromethyl trifluoromethansulfonate, [¹¹C]trifluoromethyl iodide, [¹¹C]trifluoromethyl bromide, [¹¹C]trifluoromethyl chloride, or [¹¹C]trifluoromethyl trifluoromethansulfonate. In an aspect, the foregoing labeling agent is NCA with a molar activity greater than 200 GBq/μmol, wherein the molar activity is corrected to the end of radionuclide production.

In an aspect, the generated ¹¹C-labeling agents may be described as NCA. It is to be understood, that minor radiochemical impurities arising from synthesis and incomplete purification can be present. In an aspect, the molar activity is greater than 50 GBq/μmol, specifically greater than 200 GBq/μmol.

In another aspect, a labeling agent is [¹⁸F]fluoroform, [¹⁸F]difluoromethane, [¹⁸F]difluoromethyl bromide or [¹⁸F]trifluoromethyl bromide. In an aspect, the foregoing labeling agent is NCA with a molar activity greater than 15 GBq/μmol, wherein the molar activity is corrected to the end of radionuclide production.

Specifically, to meet the objective of producing [¹¹C]fluoroform from [¹¹C]methane or [¹⁸F]fluoroform from [¹⁸F]fluoromethane, a simple, rapid, and efficient fluorination method was sought. In order to maintain high molar activity in the [¹¹C]fluoroform, for example, the method needed to be applicable to [¹¹C]methane in the presence of only a low amount of carrier methane (typically <<1 μmol) without introducing an appreciable amount of further carrier. Moreover, the method needed to be amenable to easy remote-control within a lead-shielded ‘hot-cell’ for radiation safety to personnel.

The fluorination of non-radioactive methane gas by copper(II) fluoride (CuF₂), manganese(III) fluoride (MnF₃), or iron(III) fluoride (FeF₃) at elevated temperatures (350-950° C.) has been described previously. Under all tested conditions, fluoroform was obtained in only trace yields. Other volatile gases such as ethane, ethene, and complex mixtures of volatile fluorocarbons were also common byproducts from these reactions. Byproducts were also significantly increased when dilute methane was utilized. These findings signified severe limitations for the potential use of these metal fluorides for the clean and efficient conversion of [¹¹C]methane into [¹¹C]fluoroform.

However, the present inventors noted that another metal fluoride, CoF₃ (cobalt(III) fluoride), has been used to fluorinate macro quantities of methane to give fluoroform preferentially over other fluorinated species. The CoF₃ was generated in situ by passing fluorine gas over cobalt(II) fluoride (CoF₂) held within a heated nickel tube. Fluoroform was then produced continuously by passing pure methane or 5% methane in helium through the column at a temperature of >250° C.

The present inventors considered that simple single pass of NCA [¹¹C]methane over heated CoF₃ might be an attractive possibility for producing previously unknown [¹¹C]fluoroform, provided that over-fluorination of the sub-micromole amount of carrier methane to [¹¹C]tetrafluoromethane or even to its polymers would not be a major issue, nor the formation of other hydrocarbon byproducts (possible sources of carrier) from for example apparatus materials. The inventors also considered that it would be advantageous to avoid any need for noxious and highly reactive fluorine, which can also be dangerous if inadvertently mishandled.

The inventors noted that CoF₃ is now commercially available. Therefore, the inventors considered using commercially available CoF₃ as a safe means for producing [¹¹C]fluoroform. Alternatives to CoF₃ include potassium tetrafluorocobaltate(III) (KCoF₄), and cerium tetrafluorocobaltate(III) (CeCoF₄).

The inventors designed and built a simple remotely-controllable apparatus for testing their proposal for producing [¹¹C]fluoroform. This apparatus was sufficiently compact for easy accommodation in a standard lead-shielded ‘hot-cell’ for radiochemical processing.

The inventors found that heated CoF₃ successfully mediates conversion of [¹¹C]methane into [¹¹C]fluoroform within their apparatus, which subsequently proved highly reliable and unproblematic. ¹¹C-Labeled species passed freely through the heated CoF₃.

The inventors also demonstrated the utility of the [¹¹C]fluoroform for rapid labeling of model compounds, and also examples for labeling drug-like compounds containing trifluoromethyl groups in high yields from the [¹¹C]fluoroform derivative [¹¹C]CuCF₃. These labeled compounds show very high molar activities that match those to be expected from cyclotron-produced [¹¹C]methane in the absence of further dilution with carrier. These molar activities also well exceed those previously attained for [¹⁸F]fluoroform or [¹⁸F]CuCF₃ that were generated by previously known and but unrelated methods. In particular, the molar activities with the present invention are on a par with those for PET radiotracers commonly used for imaging low density protein targets, such as neurotransmitter receptors, transporters, enzymes, and plaques.

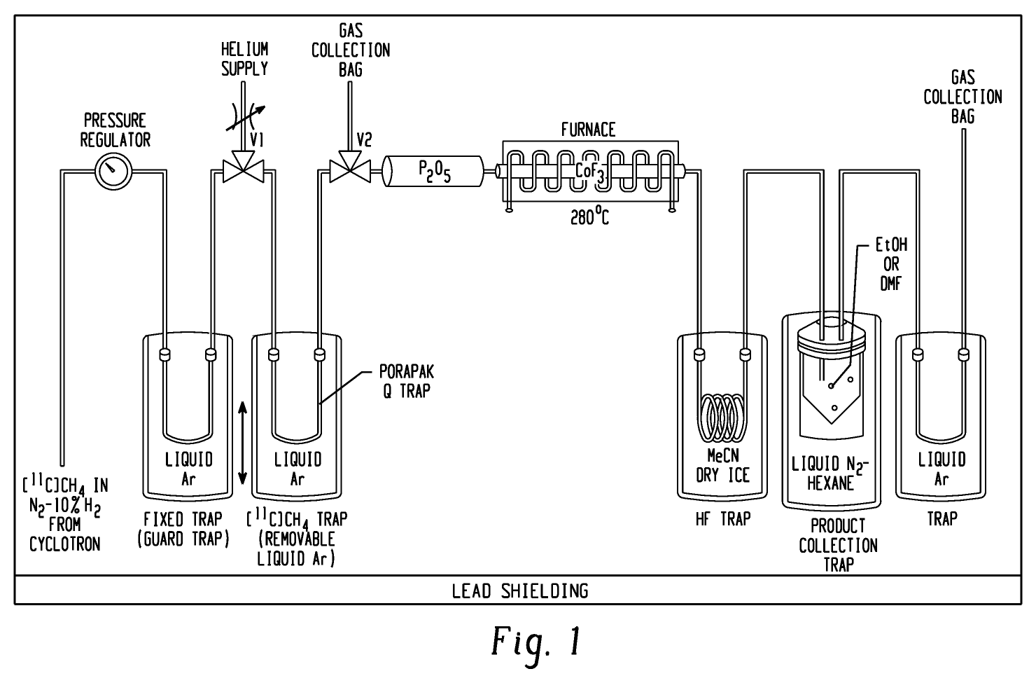

A novel aspect of the radiosynthesis of [¹¹C]fluoroform from [¹¹C]methane described herein was to use safe and commercially available CoF₃ directly as the reagent to avoid the need for generating this reagent in situ from heated cobalt(II) fluoride (CoF₂) and hazardous fluorine gas. FIG. 1 illustrates the final configuration of the apparatus that was developed for preparing [¹¹C]fluoroform.

Unexpectedly, it was found that the assembled apparatus could be used repetitively for the production of [¹¹C]fluoroform over a large number (81) of uses without any need for replacement of the CoF₃ reagent and without any decline in performance (FIG. 4). This renders the apparatus to be highly convenient to the user, as no replenishment of CoF₃ is required over a very large number of uses.

The inventors reasoned that a similar apparatus could be effective for the production of [¹⁸F]fluoroform of high molar activity, if the apparatus were charged with NCA [¹⁸F]fluoromethane that had been produced from cyclotron-produced [¹⁸F]fluoride ion, provided that all C-¹⁸F bonds would not be broken and that not all ¹⁸F radioactivity would simply bind or exchange with the heated CoF₃ or with generated hydrogen fluoride. The inventors appreciated that their new approach to the synthesis of [¹⁸F]fluoroform differed from former approaches in that the radioactive fluorine atom was the first to be introduced into the trifluoromethyl group, and that the other two non-radioactive fluorine atoms would be added subsequently under conditions in which vulnerability to C-¹⁸F or C—F bond breaking might be mitigated. The inventors reasoned that the C-¹⁸F bond in [¹⁸F]fluoromethane would be less likely to break under thermal conditions than the weaker C-H bonds. The inventors further noted that NCA [¹¹F]fluoromethane can be produced efficiently from NCA [¹⁸F]fluoride ion by several known methods, for example by treating methyl mesylate with NCA [¹⁸F]fluoride ion (approximately 32-70% yield).

It was found that [¹⁸F]fluoroform could be produced in a duplicate fluorination apparatus by feeding the apparatus with NCA [¹⁸F]fluoromethane. Although 56±6% (n=9) of the ¹⁸F radioactivity became bound to the CoF₃, the yield of [¹⁸F]fluoroform from NCA [¹⁸F]fluoromethane was still usefully high (43±6%; n=9), because as earlier mentioned [¹⁸F]fluoride ion can be produced in very high activities (approximately 1 TBq) from modem biomedical cyclotrons whereas radiotracer activities required for PET experiments are relatively very low. The inventors appreciated that the observed binding of ¹⁸F radioactivity to the CoF₃ might cause major dilution of the molar activity of [¹⁸F]fluoroform with respect to the molar activity of the starting [¹⁸F]fluoride ion. Nonetheless, this dilution was found to be very modest in practice, i.e., less than ten-fold.

In an aspect, an apparatus for gas phase production of ¹¹C- or ¹⁸F-labeled fluoroalkane from a ¹¹C- or ¹⁸F-labeled precursor comprises

an inlet for radiolabeled precursor in fluid communication with an impurity trap for removal of impurities from the radiolabeled precursor,

the impurity trap in fluid communication with a column for removal of ammonia and/or water from the radiolabeled precursor,

the column in fluid communication with a furnace suitable for heating CoF₃ to a temperature such as 50° C. to 450° C.,

the furnace optionally in fluid communication with a hydrogen fluoride trap, and

the furnace or the hydrogen fluoride trap in fluid communication with a product trap.

In an aspect, a gas phase solvent-free method for producing a ¹¹C- or ¹⁸F-labeled fluoroalkane comprises contacting [¹¹C]methane, [¹⁸F]fluoromethane, [¹¹C]fluoromethane, [¹¹C]methyl iodide, [¹¹C]methyl bromide, [¹¹C]methyl chloride, [¹¹C]methyl trifluoromethansulfonate, [¹⁸F]fluoromethyl bromide, or a combination comprising at least one of the foregoing with CoF₃ at a temperature of 50 to 450° C., and isolating the ¹¹C- or ¹⁸F-labeled fluoroalkane that is produced.

In specific embodiments:

the precursor is [¹¹C]methane and the labeled fluoroalkane is [¹¹C]fluoroform,

the precursor is [¹⁸F]fluoromethane and the labeled fluoroalkane is [¹⁸F]fluoroform,

the precursor is [¹¹C]fluoromethane and the labeled fluoroalkane is [¹¹C]difluoromethane,

the precursor is [¹⁸F]fluoromethane and the labeled fluoroalkane is [¹⁸F]difluoromethane,

the precursor is [¹¹C]methyl iodide and the labeled fluoroalkane is [¹¹C]fluoromethyl iodide,

the precursor is [¹¹C]methyl iodide and the labeled fluoroalkane is [¹¹C]difluoromethyl iodide,

the precursor is [¹¹C]methyl iodide and the labeled fluoroalkane is [¹¹C]trifluoromethyl iodide,

the precursor is [¹¹C]methyl bromide and the labeled fluoroalkane is [¹¹C]fluoromethyl bromide,

the precursor is [¹¹C]methyl bromide and the labeled fluoroalkane is [¹¹C]difluoromethyl bromide,

the precursor is [¹¹C]methyl bromide and the labeled fluoroalkane is [¹¹C]trifluoromethyl bromide,

the precursor is [¹¹C]methyl chloride and the labeled fluoroalkane is [¹¹C]fluoromethyl chloride,

the precursor is [¹¹C]methyl bromide and the labeled fluoroalkane is [¹¹C]difluoromethyl chloride,

the precursor is [¹¹C]methyl bromide and the labeled fluoroalkane is [¹¹C]trifluoromethyl chloride,

the precursor is [¹¹C]methyl trifluoromethansulfonate and the labeled fluoroalkane is [¹¹C]fluoromethyl trifluoromethansulfonate,

the precursor is [¹¹C]methyl trifluoromethansulfonate and the labeled fluoroalkane is [¹¹C]difluoromethyl trifluoromethansulfonate,

the precursor is [¹¹C]methyl trifluoromethansulfonate and the labeled fluoroalkane is [¹¹C]trifluoromethyl trifluoromethansulfonate,

the precursor is [¹⁸F]fluoromethyl bromide and the labeled [¹⁸F]fluoroalkane is [¹⁸F]difluoromethyl bromide, or

the precursor is [¹⁸F]fluoromethyl bromide and the labeled [¹⁸F]fluoroalkane is [¹⁸F]trifluoromethyl bromide.

In a specific aspect, the precursor is [¹¹C]methane or [¹⁸F]fluoromethane, and the precursor is contacted with the CoF₃ at a temperature of 260 to 290° C.

In an aspect, the precursor contains less than 3 μmol of unlabeled carrier alkane. In another aspect, contacting does not include the addition of unlabeled carrier alkane.

In an aspect, the methods further include purification of the radioactive precursor. In an aspect, the method comprises removing impurities from the precursor prior to the contacting with CoF₃. In another aspect, the method further comprises removing water and/or ammonia from the precursor prior to the contacting with CoF₃.

In an aspect, the contacting is done in a flow of helium gas or other inert gas, e.g. nitrogen, argon.

The ¹¹C- or ¹⁸F-labeled fluoroalkane that is produced can be trapped in a cold solvent trap.

In an aspect, the ¹¹C-labeled fluoroalkane that is produced is NCA. The NCA ¹¹C-labeled fluoroalkane can have a molar activity greater than 200 GBq/μmol, wherein the molar activity is corrected to the end of radionuclide production. The ¹⁸F-labeled fluoroalkane is considered according to theory to have somewhat lower molar activity than the starting [¹⁸F]fluoromethane, due to fluorine for ¹⁸F-fluorine exchange with hydrogen fluoride and/or CoF₃, but this molar activity is still moderately high. In an aspect, the molar activity is comparable to or greater than the molar activity of [¹⁸F]fluoroform produced by former methods.

In an aspect, the radioactive precursor is either [¹¹C]methane or [¹⁸F]fluoromethane, and the radioactive precursor is contacted with the CoF₃ at a temperature of 260 to 290° C.

In an aspect, a method of preparing an ¹¹C-labeled or ¹⁸F-labeled radiotracer, comprises combining [¹¹C]fluoroform or [¹⁸F]fluoroform with a non-radioactive precursor to form a reaction mixture, and producing the ¹¹C-labeled radiotracer or the ¹⁸F-labeled radiotracer from the reaction mixture, wherein the non-radioactive precursor contains a functionality that is reactive with [¹¹C]fluoroform or [¹⁸F]fluoroform.

In another aspect, a method of preparing an ¹¹C-labeled or ¹⁸F-labeled radiotracer comprises converting [¹¹C]fluoroform or [¹⁸F]fluoroform into [¹¹C]CuCF₃ or [¹⁸F]CuCF₃, combining the [¹¹C]CuCF₃ or [¹⁸F]CuCF₃ with a non-radioactive precursor to form a reaction mixture, and producing the ¹¹C-labeled radiotracer or the ¹⁸F-labeled radiotracer from the reaction mixture, wherein the non-radioactive precursor contains a functionality that is reactive with the [¹¹C]CuCF₃ or [¹⁸F]CuCF₃.

Exemplary nonradioactive precursors include a diaryl ketone, a diaryl disulfide, an arylcarboxylic ester, an arylboronic acid, an aryl iodide, an aryldiazonium salt, a diaryliodonium salt, or an aryl(vinyl)iodonium salt.

In an aspect, functionalities that are reactive with [¹¹C]fluoroform or, [¹⁸F]fluoroform, include ketones and disulfides, in particular diaryl ketones and diaryl disulfides.

In an aspect, the non-radioactive precursor is a ketone, preferably a diaryl ketone, or a disulfide, preferably a diaryl disulfide.

In another aspect, a method of preparing a labeled compound comprises combining a [¹¹C]fluoroform or a [¹⁸F]fluoroform solution with a carbonyl compound solution to form a reaction mixture, and isolating the labeled compound.

In another aspect, a method of preparing a labeled compound comprises combining a [¹¹C]fluoroform or [¹⁸F]fluoroform solution with a diaryl disulfide solution containing potassium t-butoxide and cesium fluoride at room temperature (RT) to 180° C. to form a reaction mixture, and isolating the labeled compound.

In another aspect, a method of preparing [¹¹C]CuCF₃ comprises treating [¹¹C]fluoroform with potassium t-butoxide and copper(I) bromide or copper(I) chloride in dimethyl formamide (DMF), optionally in the presence of other mediators, such as Et₃N.3HF.

In another aspect, a method of preparing [¹⁸F]CuCF₃ comprises treating [¹⁸F]fluoroform with potassium t-butoxide and copper(I) bromide or copper(I) chloride in DMF optionally in the presence of other mediators, such as Et₃N.3HF.

In an aspect, functionalities that are reactive toward [¹¹C]CuCF₃ or [¹⁸F]CuCF₃ include arylboronic acids, aryl iodides, aryldiazonium salts, diaryliodonium salts, and aryl(vinyl)iodonium salts.

In an aspect, the non-radioactive precursor is an arylboronic acid, aryl iodide, aryldiazonium salt, diaryliodonium salt, or aryl(vinyl)iodonium salt.

In another aspect, a method of preparing a labeled compound comprises combining a [¹¹C]CuCF₃ or [¹⁸F]CuCF₃ solution with an arylboronic acid, aryl iodide, aryldiazonium salt, diaryliodonium salt, or aryl(vinyl)iodonium salt solution to form a reaction mixture and isolating the labeled compound from the reaction mixture.

In another aspect, a labeled compound is prepared by combining Cu¹¹CF₃ or [¹⁸F]CuCF₃ with an arylboronic acid in a solvent at a temperature of 0 to 100° C.

In another aspect, a labeled compound is prepared by combining Cu¹¹CF₃ or [¹⁸F]CuCF₃ with an aryl iodide in a solvent at a temperature of 0 to 180° C.

In another aspect, a radiolabeled compound is prepared by combining Cu¹¹CF₃ or [¹⁸F]CuCF₃ with an aryldiazonium salt in a solvent at a temperature of 0 to 100° C.

In another aspect, a radiolabeled compound is prepared by combining Cu¹¹CF₃ or [¹⁸F]CuCF₃ with a diaryliodonium salt in a solvent at temperature of RT (approximately 23° C.) to 200° C.

In another aspect, a radiolabeled compound is prepared by combining Cu¹¹CF₃ or [¹⁸F]CuCF₃ with an aryl(vinyl)iodonium salt in a solvent at a temperature of RT to 200° C.

In certain cases these molecules may be used as radiotracers, following purification and formulation procedures that are generally well known to those practiced in the art.

The radiotracers produced using the labeling agents described herein may be used in nuclear medicine molecular imaging techniques, such as positron emission tomography (PET), for example wherein 511 keV gamma radiation is detected. Typically, for nuclear medicine imaging, the radiation emitted in a subject from an organ or an area being examined is measured and followed over time with a suitable detection system, such as a “PET camera.” A “subject” is a mammal, such as a human. These data in combination with other collected data (such as a radiometabolite-corrected arterial input function) may be used to derive useful output measures such as total volumes of distribution for the radiotracer, or the density of a protein target to which the radiotracer binds.

Radiotracers labeled with carbon-11 or fluorine-18 are particularly suitable for in vivo PET imaging. The half-life of the radionuclide used for labeling the radiotracer is an important consideration. The half-life should be long enough so that it is still detectable at the time of maximum uptake by the target and for a period beyond, but short enough so that the host does not sustain deleterious radiation. Radiotracers labeled with carbon-11 are particularly useful for imaging sites in close proximity to the facility for producing the carbon-11 radionuclide. This is because the short half-life may allow more than one injection of the radiotracer into the same animal or human subject in the same day. Separation of the two scanning sessions by a few hours allows virtually full radioactive decay between the two injections. These injections may be performed under different but controlled physiological or pharmacological circumstances to assess the influence of these conditions on radiotracer behavior or output measures. By contrast, radiotracers labeled with fluorine-18 at high activity may be transported for several hours from the site of production and potentially to several imaging centers before use. Indeed, such transportation of [¹⁸F]fluoride ion or of derived radiotracers is regularly performed on a commercial basis. However, generally a subject may only be injected with an ¹⁸F-labeled radiotracer once in a day for a single scanning session. Nonetheless, fluorine-18 radioactivity may be measured in the subject over longer periods than carbon-11 radioactivity, and consequently may provide additional information.

The ability to label the same radiotracer with carbon-11 or fluorine-18 in the same metabolically stable group provides potential to use a radiotracer for local use with a carbon-11 label and for remote use with a fluorine-18 label, with generation of highly comparable imaging results, and with facilitation of sharing of expensively or difficultly acquired PET imaging data. The [¹¹C]fluoroform described here, together with [¹⁸F]fluoroform provides for this attractive possibility, because each of these agents may be used to label metabolically stable trifluoromethyl groups by the same rapid and efficient radiochemical processes.

Generally, the dosage of the labeled radiotracer (predominantly consisting of its accompanying non-radioactive carrier) will vary depending on considerations such as age, condition, sex, and extent of disease in the patient, contraindications, if any, concomitant therapies and other variables, to be adjusted by a physician skilled in the art. Dosage can vary from 0.001 μg/kg to 10 μg/kg, specifically 0.01 μg/kg to 1.0 μg/kg.

Administration to the subject can be local or systemic and accomplished intravenously, intra-arterially, intrathecally (via the spinal fluid) or the like. Administration can also be intradermal or intracavitary, depending upon the body site under examination. After administration of the radiotracer, the exact imaging protocol can vary depending upon factors specific to the subject, as noted above, and depending upon the body site under examination, method of administration and type of label used; the determination of specific procedures would be routine to the skilled artisan. Blood sampling may accompany imaging to allow for measurement of the radiometabolite-corrected arterial input function of the radiotracer. These PET and blood measurements can then be used by well-known biomathematical techniques to quantify radiotracer density in areas of interest.

Examples of non-aqueous carriers for the radiotracer are propylene glycol, polyethylene glycol, vegetable oil, and injectable organic esters such as ethyl oleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobials, anti-oxidants, chelating agents and inert gases. The pH and exact concentration of the various components of the pharmaceutical composition are adjusted according to routine skills in the art.

The invention is further illustrated by the following non-limiting examples.

### EXAMPLES

### Example 1. Construction, Set-Up, and Operation of Apparatus for [¹¹C]Fluoroform Synthesis

### Construction.

General Materials and Methods.

The apparatus shown in FIG. 1 was constructed, set-up, and operated as follows. A gas drying trap (200 cc) was purchased from Agilent (Santa Clara, Calif.). PORAPAK™ Q (80-100 mesh) was purchased from Supelco (Bellefonte, Pa.). Prewashed stainless steel tubing (0.21 inch (″) I.D.; 0.25″ O.D.), stainless steel tubing (0.085″ I.D.; 0.125″ O.D.), and quartz wool were purchased from GRACE (Deerfield, Ill.). Seamless stainless steel tubes (316/316L; 0.375″ O.D.; 0.035″ wall thickness; 14.5″ length) were obtained from Swagelok (Allentown, Pa.). All compression fittings were purchased from Swagelok. Stainless steel frits (2 m pore size; 0.375″ diameter) were purchased from VICI (Houston, Tex.). Polytetrafluoroethylene (PTFE) tubing (0.062″ I.D.; 0.125″ O.D) was obtained from GE Medical Systems (Milwaukee, Wis.). A gas flow controller was obtained from Alicat Scientific (Tucson, AS). Miniature 3-way valves (Series 9 microdispense) were obtained from Parker (Hollis, N.H.).

[¹¹C]Methane Guard Trap.

Stainless steel tubing (0.21″ I.D.; 0.25″ O.D.; 11.5″ length) was bent into a U-shape (2.2″ wide; 5″ length). Both ends of the U-tube were connected to stainless steel tubing (0.085″ I.D.; 0.125″ O.D.) with reducing unions (0.25″ to 0.125″), with one tube connecting to a pressure regulator and the other to a 2-way valve (FIG. 1).

[¹¹C]Methane Trap.

A second trap was constructed in the same way as the guard trap and filled with PORAPAK™ Q (1.0 g; 80-100 mesh). Each end was plugged with quartz wool and then fitted with a stainless steel frit (2 m pore size) and a reducing union (0.25″ to 0.125″). The inlet of this trap was connected with PTFE tubing (0.062″ I.D.; 0.125″ O.D) to the 2-way valve placed on the outlet side of the guard trap. The outlet was connected to another 2-way valve (FIG. 1).

Sicapent® Column.

[¹¹C]Methane may contain traces of ammonia (and/or water if [¹¹C]methane is produced from [¹¹C]carbon dioxide using nickel catalyst and hydrogen gas). Such residual contaminants are ideally removed from [¹¹C]methane before contact with CoF₃. Sicapent® (phosphorus pentoxide on a neutral indicator resin) was used for this purpose. Thus, one end of a stainless steel tube (0.305″ I.D.; 0.375″ O.D.; 10″ length) was plugged with quartz wool (about 1″) and then Sicapent® was added. Then the other end of the tube was likewise plugged with quartz wool. Both ends were then fitted with a stainless steel frit (2 m pore size, 0.375″ diameter) and a reducing union (0.375″ to 0.125″). One end of this column was then connected to a 2-way valve and the other to the CoF₃ column (FIG. 1).

Cof₃ Column.

A stainless steel tube (0.305″ I.D.; 0.375″ O.D.; 14.5″ length) was dried in a hot (110° C.) oven overnight and then transferred into a glovebox (having dry nitrogen atmosphere) and allowed to cool. One end was plugged with quartz wool (1″), and then CoF₃ (18.8±0.4 g) was dispensed into the column through a dry glass funnel. The open end of the tube was then plugged with quartz wool (1″). One end of the column was fitted with a stainless steel frit (2 m pore size, 0.375″ diameter) and a reducing union (0.375″ to 0.125″). The other end was closed with Parafilm®. The column was then taken out of the glovebox and, after removal of the Parafilm®, was quickly inserted into the furnace. The open end of the column was quickly sealed with another frit and reducing union. One end of the column was then connected quickly to the Sicapent® column and the other to the HF trap described below, each with stainless steel tubing (0.125-inch O.D.).

Hf Trap.

In experiments in which [¹¹C]fluoroform was to be used for labeling reactions, the effluent from the CoF₃ column was passed through a coil (2.5″ loop diameter) of stainless steel tubing (0.085″ I.D.; 0.125″ O.D.; 25″ length) that was immersed in acetonitrile-dry ice (approximately −41° C.). This trap served to cool the effluent and to remove any traces of hydrogen fluoride (HF; b.p. 19.5° C.) before subsequent trapping of radioactive products.

Radioactive Product Collection Trap.

Radioactive products were collected from the helium gas stream in ethanol (5 mL) cooled with a hexane/liquid nitrogen bath (approximately −94° C.) or DMF (1 mL) cooled with an acetonitrile-dry ice bath (approximately −41° C.). These traps retained most of the [¹¹C]fluoroform (b.p. −82.1° C.). They were also expected to retain most of any likely labeled byproducts, such as [¹¹C]fluoromethane (b.p. −78.4° C.) or [¹¹C]difluoromethane (b.p. −52° C.).

### Apparatus Set-Up and Operation.

Immediately after insertion of the CoF₃ column, the whole apparatus was flushed with helium for about 30 min. The flow of helium out of the apparatus was measured with a flow meter (Agilent Technologies; Santa Clara, Calif.) to verify that this was within 0.2 mL/min of the set point of the gas flow controller. The apparatus was leak-checked by capping the gas outlet (0.125″ cap; Swagelok) while leaving the apparatus open to helium pressure (70 psi). The apparatus was considered leak-tight if the flow controller showed zero flow within 10 min. The system was then left fully closed and filled with helium until used.

Before any experiment, the two U-tube traps were immersed in liquid argon for 15 min. The empty U-tube trap was kept immersed throughout the experiment. Gas released from the irradiated cyclotron target was held at 55 psi with a pressure regulator (100 psi; Swagelok).

[¹¹C]Methane was produced by the 14N(p,α)¹¹C nuclear reaction by irradiation of nitrogen gas (164 psi) containing hydrogen (10%) with a proton beam (16.5 MeV; 5-45 μA) generated with a PETtrace™ 200 cyclotron (GE Healthcare; Milwaukee, Wis.) for 3 to 40 min.

[¹¹C]Methane was trapped by passing gas from the irradiated cyclotron target through both cooled U-traps and out into a bag for the safe collection of the nitrogen-hydrogen gas mixture and any untrapped radioactivity. When radioactivity in the cooled PORAPAK™ Q U-tube trap had maximized, as indicated by a nearby radiation detector, the liquid argon coolant was removed. The trap was then allowed to warm to RT under a controlled helium flow to direct [¹¹C]methane successively over the Sicapent® and heated CoF₃ columns, through the cooled HF trap and into the cooled product collection trap. The CoF₃ column was preferably operated with a helium flow rate of 20 mL/min and with an oven temperature of 270° C.

### Analysis of Apparatus Performance

Determination of the Efficiency of [¹¹C]Methane Trapping.

Two U-shaped [¹¹C]methane traps were filled with equal amounts of PORAPAK™ Q material. The two traps were then connected in tandem with PTFE tubing (0.0625″ I.D.; 0.125″ O.D.) with reduction fittings (0.25″ to 0.125″). The outlet of the second trap was connected to a gas collection bag via PTFE tubing (0.0625″ I.D.; 0.125″ O.D.). Both traps were immersed in liquid argon (−186° C.) at 15 minutes (min) before trapping experiments were initiated. The trapping efficiency of [¹¹C]methane (%) was calculated as radioactivity trapped in the first trap divided by the sum of radioactivity trapped in both traps, multiplied by 100. Results are shown in Table 1.

TABLE 1
Dependence of [¹¹C]methane trapping efficiency and
release time on quantity of PORAPAK ™ Q

| | | Trapping | Release- |
|---|---|---|---|
| | PORAPAK ™ Q | efficiency^{a} | time |
| Entry | (g) | (%) | (min) |
| 1 | 0.2 | 53.0 ± 12.0 | 3 |
| 2 | 0.4 | 59.3 ± 7.6 | 3 |
| 3 | 1.0 | 98.9 ± 0.1 | 4 |

^{a}n = 3

The conditions in entry 3 were deemed optimal.

Early experiments showed that [¹¹C]methane readily passed through the CoF₃ column without any hold-up of radioactivity or any excessive restriction of flow, and with some conversion into [¹¹C]fluoroform at elevated temperature.

Temperature Dependence of [¹¹C]Fluoroform Synthesis.

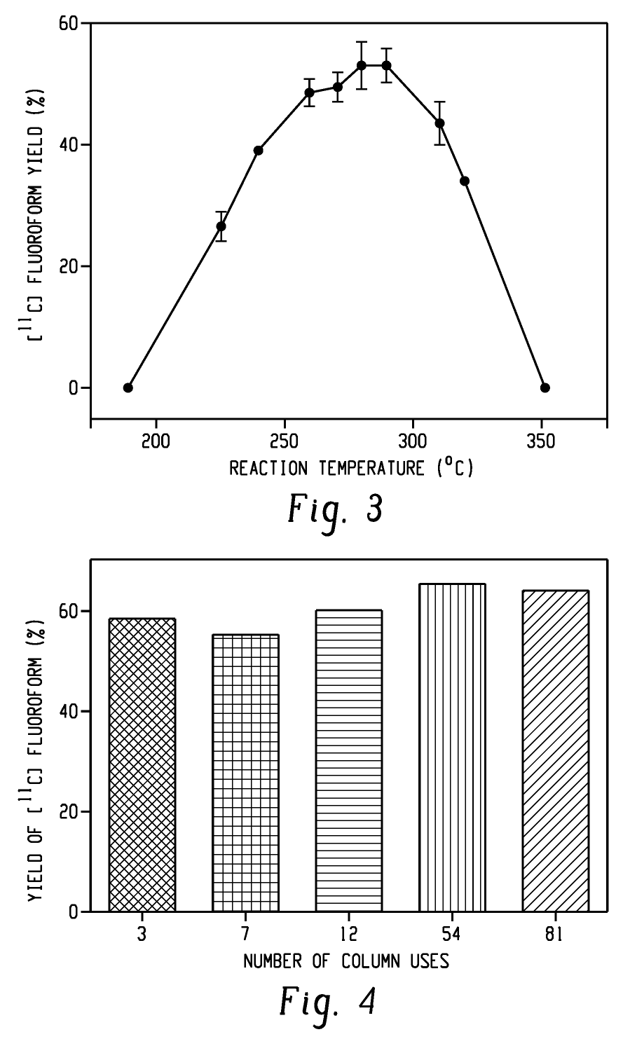

Initially, the temperature dependence of the conversion of [¹¹C]methane into [¹¹C]fluoroform was investigated with the flow of carrier helium set at an optimal value of 20 mL/min. Only radioactivity trapped in cold (approximately −94° C.) ethanol solution was used to calculate the radiochemical yield (the breakthrough of radioactivity into a subsequent trap was relatively low). High yields of [¹¹C]fluoroform were obtained between 260 and 290° C., with 280° C. appearing optimal (FIG. 3). The average yield of [¹¹C]fluoroform in the 260 to 290° C. temperature range was 53±4%, based on 11 experiments conducted with two different CoF₃ columns.

The overall process for producing [¹¹C]fluoroform from cyclotron-produced [¹¹C]methane required only 7 minutes from the end of a cyclotron irradiation, and was thus much less than one half-life of carbon-11.

Repeatability of CoF₃ Column Performance.

Whereas heating of a CoF₃ column to 350° C. multiple times resulted in severely impaired performance, initial conditioning of a newly installed CoF₃ column by heating it once to 350° C. resulted in optimal yields of [¹¹C]fluoroform at lower temperatures. Moreover, such a heat-conditioned CoF₃ column showed remarkably consistent performance over a large number of [¹¹C]fluoroform productions; in fact, the yields of [¹¹C]fluoroform showed no tendency to diminish over 81 runs (FIG. 4). Therefore, once set up and conditioned, the apparatus required no significant maintenance other than to be kept filled with helium. Also the apparatus was very simply adapted for automation and remotely control to ensure radiation protection to personnel.

The robustly repeatable performance of a conditioned CoF₃ column suggests that the fluorination process does not depend on prior decomposition of the CoF₃ to CoF₂ plus fluorine. Without being held to theory, it is believed that the [¹¹C]methane interacts directly and stoichiometrically with CoF₃ to remove one fluorine atom to first give [¹¹C]fluoromethane, which then repeats the fluorine atom abstraction until [¹¹C]fluoroform is formed.

Calculation of Recovery of Radioactivity from the Apparatus.

The percentage recovery of radioactivity was calculated as the sum of radioactivity in the cooled ethanol trap plus the subsequent liquid argon cooled trap divided by the radioactivity trapped in the PORAPAK™ Q trap before [¹¹C]methane release, multiplied by 100. Recovery was estimated to be 86±6%.

Calculation of Conversion of [¹¹C]Methane into Other Radioactive Products.

The percentage conversion of [¹¹C]methane into radioactive products that were trapped in ethanol was calculated by dividing the radioactivity collected in the ethanol trap by the initial radioactivity in the [¹¹C]methane trap, multiplied by 100. Conversion was estimated to be 72±7%.

Analysis of ¹¹C-Labeled Reaction Products.

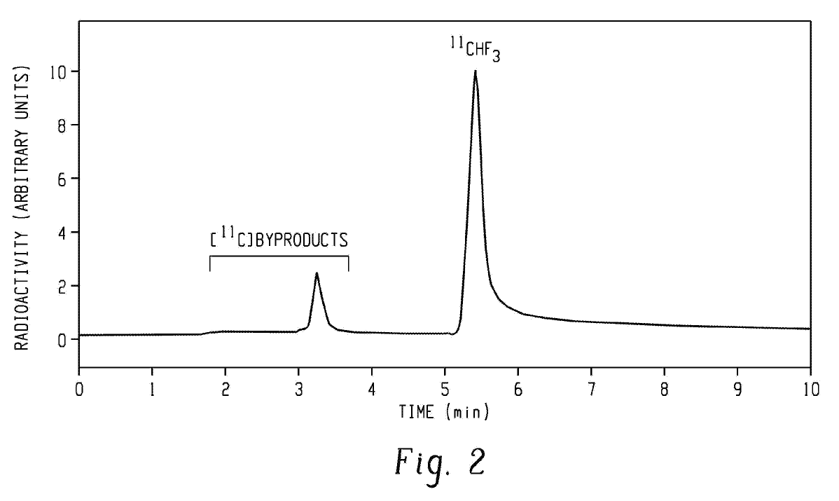

The radioactivity trapped in cold ethanol solution was analyzed by radio-high pressure liquid chromatography (HPLC) on a Luna® C18 column (10 am, 100 Å, 250×4.60 mm) eluted at 2 mL/min with a gradient of MeCN (A)-0.1% (w/v) HCOONH₄ (B), started at 5% A for 1 min, increased linearly to 90% A over 10 min, and maintained at 90% A for 14 min. Typical injection volumes were 20-50 μL. The eluate was monitored for radioactivity with a scintillation detector (Bioscan). The radioactive products eluted between 1 and 3.5 min, and at 5.3±0.2 min (FIG. 2).

Identification of [¹¹C]fluoroform.

An experiment was designed to verify the identity of the peak seen at 5.3 min in radio-HPLC as [¹¹C]fluoroform. First, a balloon was inflated with fluoroform gas. This fluoroform was then bubbled into the cold ethanol solution containing radioactive products. A fraction (approximately 50 μL) of this material was analyzed with radio-HPLC under the aforementioned conditions. Peaks eluting between 1-3.2 min and appearing at 5.3±0.2 min were collected separately in septum-sealed 1.5-mL vials (12×32 screw cap; Agilent Technologies; Santa Clara, Calif.). These samples were sonicated and the headspace gas (2 μL) was analyzed with GC-MS on a PolarisQ™ instrument (Thermo Fisher Scientific; Waltham, Mass.) using a Restek Rtx®-5MS column (0.25 mm ID×30 m) at 100° C. with a 1 mL/min helium flow rate. Acquisition was set to electron spray (EI) mode with the ion source temperature set at 200° C. for 5 min. A mass range of 50-150 amu was recorded. The major radioactive peak appearing at 5.3±0.2 min was confirmed to be [¹¹C]fluoroform, as the headspace gas gave the same mass spectrum as authentic fluoroform (m/z=69 (100%), [CF₃]+; 51.2 (64%), [CHF₂+H]⁺; 50.2 (10%), [CHF₂]+, 32 (25%), [CHF]⁺. No fluoroform was detected in the GC-MS analysis of products eluting between 1 and 4 min. These products are likely [¹¹C]fluoromethane and [¹¹C]difluoromethane.

Yield Calculation for [¹¹C]fluoroform.

The percentage of radioactivity represented by [¹¹C]fluoroform in the HPLC analyte was calculated from the radio-HPLC chromatogram from peak areas with a small correction for radioactive decay between radioactive peaks during the analysis. This correction was multiplication of the [¹¹C]fluoroform peak area by 1.08 to allow for 2.2 min of decay between the early radioactive peaks and the [¹¹C]fluoroform peak. Radioactivity trapped in the cold ethanol was found to be the major portion of radioactivity that had been introduced initially into the apparatus. Therefore, the yield of [¹¹C]fluoroform was calculated as the percentage of radioactivity represented by [¹¹C]fluoroform in the HPLC analyte multiplied by the percentage recovery of initial radioactivity recovered in the cold ethanol. Table 1 shows the dependence of [¹¹C]methane trapping efficiency and release-time on quantity of PORAPAK™ Q.

### Example 2. Conversion of [¹¹C]Fluoroform to [¹¹C]CuCF₃ and Uses

Conversion of [¹¹C]fluoroform into [¹¹C]CuCF₃. Within a glovebox having a dry nitrogen atmosphere, potassium tert-butoxide (t-BuOK) in DMF (0.3 M, 50 μL) was added to CuBr (0.7 mg, 5 μmol) in a dry 1-mL glass vial. The vial was septum-sealed and removed from the glovebox. [¹¹C]Fluoroform in DMF (50-300 μL) was added to the vial, mixed, and left at RT for 1 min. A solution of Et₃N.3HF in DMF (1.64% v/v; 50 μL) was then added. The mixture was mixed thoroughly and allowed to stay at RT for another minute, before use in labeling reactions.

Radiochemistry with [¹¹C]Fluoroform and the Derivative [¹¹C]CuCF₃

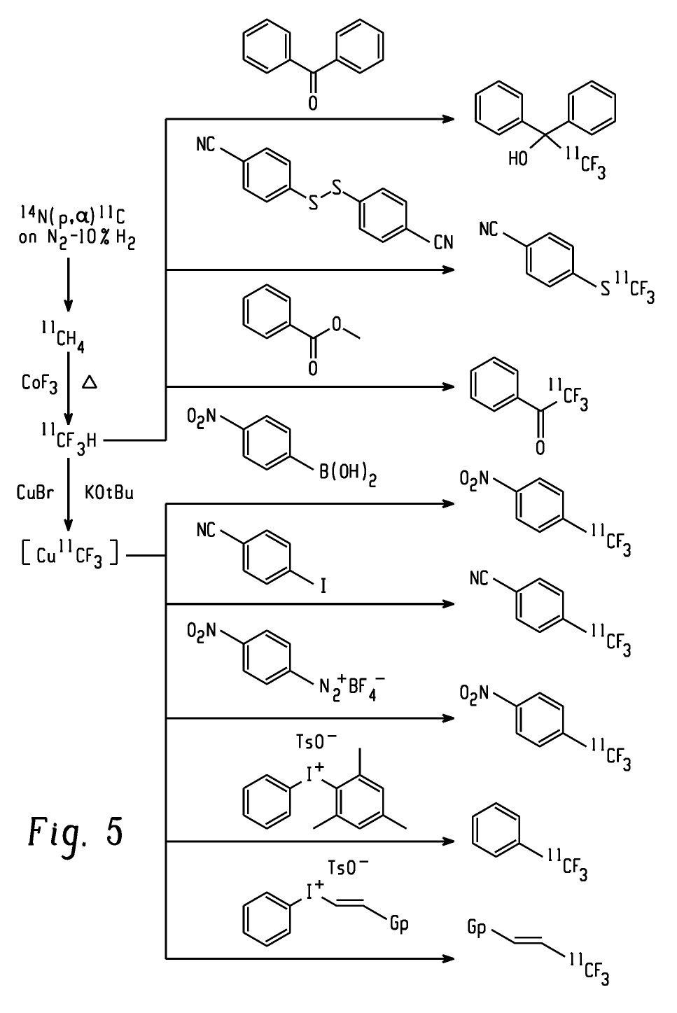

The following represent general methods for the uses of [¹¹C]fluoroform and the derivative [¹¹C]CuCF₃ for compound labeling. The chemistry of these general methods is summarized in FIG. 5. Generally, reactions were tested with 185-555 MBq of labeling agent. The non-radioactive compounds for labeling may be variously substituted or appended with other groups including functional groups, as shown in some later specific examples, including labeled drugs.

### General ¹¹C-Labeling Methods

Preparation of a [¹¹C]Trifluoromethylarene from [¹¹C]Fluoroform and a Diaryldisulfane ((ArS)₂).

Within a glovebox, freshly prepared t-BuOK (0.3 M) in DMF (50 μL) and CsF (2-6 mg; 13-40 μmol) were septum-sealed within a dry 1-mL glass vial. [¹¹C]Fluoroform in DMF (50-300 μL) was then added. A diaryl disulfane (100 μmol) in DMF (0.1 mL) was then added and heated to 100° C. for 10 min.

Preparation of a [¹¹C]Trifluoromethylarene from [¹¹C]CuCF₃ and an Arylboronic Acid.

A solution of an arylboronic acid (50 μmol) in DMF (100 μL) was added to [¹¹C]CuCF₃ in DMF (approximately 0.2 mL) within a dry septum-sealed glass vial. Air (10 mL) was them bubbled through the mixture at RT.

Preparation of a [¹¹C]Trifluoromethylarene from [¹¹C]CuCF₃ and an Aryl Iodide.

A solution of aryl iodide (100 μmol) in DMF (100 μL) was added to [¹¹C]CuCF₃ in DMF (approximately 0.2 mL) within a dry septum-sealed glass vial, heated to 130° C. for 5 min, and then cooled to RT.

Preparation of a [¹¹C]Trifluoromethylarene from [¹¹C]CuCF₃ and an Aryl Diazonium Salt.

A solution of diazonium salt (50 μmol) in DMF (100 μL) was added to freshly prepared [¹¹C]CuCF₃ in DMF (approximately 0.2 mL) within a dry septum-sealed glass vial and left for 10 min at RT.

Preparation of a [¹¹C]Trifluoromethylarene from [¹¹C]CuCF₃ and a Diaryliodonium Salt.

A solution of diaryliodonium salt (50 μmol) in DMF (100-200 μL) was added to [¹¹C]CuCF₃ in DMF (approximately 0.2 mL) within a dry septum-sealed glass vial and left for 10 min at RT.

### Specific Examples of the Application of [¹¹C]Fluoroform to Labeling Model Compounds in Trifluoromethyl Groups

Labeled Product Analysis.

All radioactive products from reactions of [¹¹C]fluoroform or [¹¹C]CuCF₃ (except those from reactions with diaryliodonium salts) were quenched with MeCN-H₂O (3:1 v/v; 5 mL or 10 mL) and analyzed with reversed phase HPLC on a Luna C18 column (10 jam; 250×4.6 mm) eluted at 2 mL/min with a gradient of MeCN (A)-H₂O (B), with A started at 45% (v/v) for 1 min, increased linearly to 65% over 10 min and kept at 65% for 5 min.

Labeled products from diaryliodonium salts were quenched with MeCN-0.1% aqueous TFA (1:1, 5 mL), filtered through a syringe filter (0.2 am, PTFE) and analyzed with HPLC on column (Luna® C18, 10 am, 250×10 mm) eluted at 2 mL/min with a mobile phase gradient of 0.1% aqueous TFA (A) and MeCN (B) with A started at 55% for 1 min, increased linearly to 80% over 10 min and kept at 80% for another 5 min.

Eluates were monitored for absorbance at 254 nm and radioactivity with a scintillation detector (Bioscan; Washington D.C.). The identities of labeled products were confirmed by their comobilities with authentic reference compounds.

Molar Activity Determination.

The radio-HPLC system for analysis of [¹¹C]fluoroform reaction products was calibrated for UV absorption response versus mass of reference compound in the analyte. This allowed the mass of carrier in the injectate to be calculated and the molar activity to be calculated from the additional measurement of radioactivity in the labeled compound peak. The molar activity was decay-corrected to the end of radionuclide production.

### Example 3. Preparation of [¹¹C]2,2,2-trifluoro-1,1-diphenylethanol from [¹¹C]fluoroform

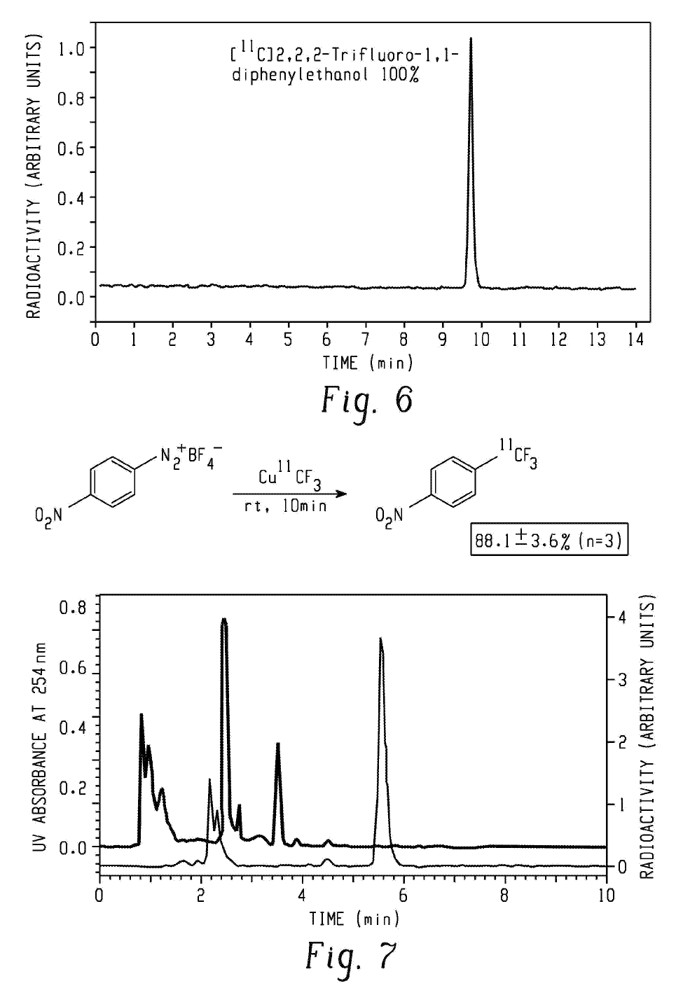

Within a glovebox, t-BuOK (5 mg, 44 μmol) and benzophenone (10 mg, 54.9 μmol) were placed in a small 1-mL vial. DMF (0.4 mL) was added and the vial was septum-sealed and removed from the glovebox. [¹¹C]Fluoroform in DMF (50-300 μL) was then added, mixed, and left at RT for 5 min. [¹¹C]2,2,2-Trifluoro-1,1-diphenylethanol was obtained quantitatively (n=3). FIG. 6 shows the radiochromatogram from the analysis of the reaction mixture.

### Example 4. Preparation of [¹¹C]Trifluoromethylthioarenes from Diaryl Disulfanes

The earlier general method described for reactions of [¹¹C]CuCF₃ with diaryl disulfanes gave the results summarized in Table 2.

TABLE 2
Yields of [¹¹C]trifluoromethylthioarenes from reactions of diaryl
disulfanes with [¹¹C]fluoroform.

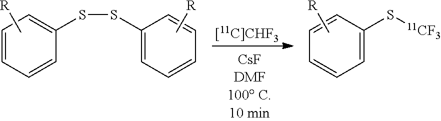

| Entry | R | Yield of ¹¹C-labeled product (%) |
|---|---|---|
| 1 | H | 51.9 |
| 2 | 4-Me | 89 ± 3 (n = 2) |
| 3 | 4-MeO | 93.6 |
| 4 | 4-CN | 29 ± 1 (n = 3) |

Loss of radioactivity from the heated reaction vial was determined to be <10%.

### Example 5. Preparation of [¹¹C]1-nitro-4-(trifluoromethyl)benzene

Treatment of 4-nitrophenylboronic acid with [¹¹C]CuCF₃ for 1 min at RT gave [¹¹C]1-nitro-4-(trifluoromethyl)benzene in 99±1% yield (n=3) and with a molar activity of 551 GBq/μmol.

### Example 6. Preparation of [¹¹C]1-fluoro-3-(trifluoromethyl)benzene

Treatment of 3-fluorophenylboronic acid with [¹¹C]CuCF₃ gave [¹¹C]1-fluoro-3-(trifluoromethyl)benzene in 98±1% yield (n=3) and with a molar activity of 242 GBq/μmol. Together, the results from Examples 5 and 6 confirmed that [¹¹C]fluoroform was produced from cyclotron-produced [¹¹C]methane without appreciable dilution of molar activity. In this example, the molar activity was over 20-fold higher than that of [¹⁸F]fluoroform prepared by earlier reported methods.

### Example 7. Preparation of ethyl [¹¹C]4-(trifluoromethyl)benzoate

Treatment of ethyl 4-iodobenzoate with [¹¹C]CuCF₃ gave ethyl [¹¹C]4-(trifluoromethyl)benzoate in 88±2% yield (n=3).

### Example 8. Preparation of [¹¹C]4-(trifluoromethyl)benzonitrile

Treatment of 4-iodobenzonitrile with [¹¹C]CuCF₃ gave [¹¹C]4-(trifluoromethyl)benzonitrile in 90±4% yield (n=3).

### Example 9. Preparation of [¹¹C](Trifluoromethyl)Arenes from [¹¹C]Cu¹¹CF₃ and Aryldiazonium Salts

The earlier general method described for reactions of [¹¹C]CuCF₃ with aryldiazonium salts gave the results summarized in Table 3.

TABLE 3
Yields of [¹¹C]trifluoromethylarenes from reactions of aryldiazonium
salts with [¹¹C]CuCF₃.

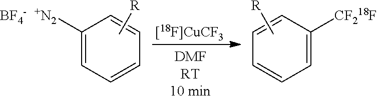

| Entry | R | Yield of ¹¹C-labeled product (%) (n = 2) |
|---|---|---|
| 1 | 4-NO₂ | 92.5 ± 5.3 |
| 2 | 4-MeO | 95.2 ± 1.2 |
| 3 | 4-Br | 85.3 ± 1.9 |
| 4 | 3,5-di-Cl | 29.5 ± 3.9 |

FIG. 7 shows the radiochromatogram for the reaction mixture from the radiosynthesis in entry 4 of Table 3.

### Example 10. Preparation of [¹¹C](Trifluoromethyl)Benzene from Mesityl(Phenyl)Iodonium Tosylate

The general procedure for reaction of [¹¹C]CuCF₃ with diaryliodonium salts when applied to mesityl(phenyl)iodonium tosylate gave [¹¹C](trifluoromethyl)benzene in 96% yield. This example shows that the mesityl group is an effective spectator group in these reactions, i.e., a group which does not undergo extensive ¹¹C-trifluoromethylation, resulting in highly selective ¹¹C-trifluoromethylation of the opposite aryl group. The results obtained by varying reaction parameters are shown in Table 4.

TABLE 4
Dependence of yield of [¹¹C](trifluoromethyl)benzene from [¹¹C]CuCF₃ and
mesityl(phenyl)iodonium tosylate on amount of diaryliodonium salt, temperature and
reaction time.

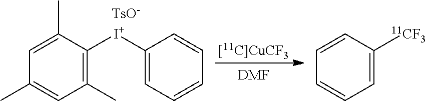

| | Precursor mount | Reaction | Reaction | Yield |
|---|---|---|---|---|
| Entry | (μmol) | temperature (° C.) | time (min) | (%) |
| 1 | 50 | RT | 5 | 63 |
| 2 | 5 | RT | 10 | 12 |
| 3 | 10 | RT | 10 | 26 |
| 4 | 25 | RT | 10 | 69 |
| 5 | 50 | RT | 10 | 96 |
| 6 | 5 | 50 | 5 | 5 |
| 7 | 10 | 50 | 5 | 43 |
| 8 | 25 | 50 | 5 | 60 |
| 9 | 50 | 50 | 5 | 99 |

The mild conditions in entry 5 were selected for general application.

### Example 11. Preparation of Substituted [¹¹C]Trifluoromethylarenes

Table 5 shows the yields of substituted [¹¹C]trifluoromethylarenes obtained from mesityl(aryl)iodonium tosylates and [¹¹C]CuCF₃ performed under the reaction conditions of Example 10, Table 4, entry 5 (diaryliodonium salt (50 μmol); DMF (100-200 μL); [¹¹C]CuCF₃ in DMF (approximately 0.2 mL); 10 min; RT). These are non-limiting examples exemplifying the scope of the reaction.

TABLE 5
Yields from the reactions of mesityl(aryl)iodonium tosylates with
[¹¹C]CuCF_{3.}

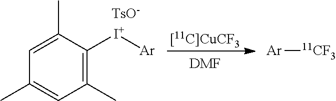

| Entry | Ar | Yield of Ar—¹¹CF₃ (%) | |
|---|---|---|---|
| 1 | Ph | 94.8 ± 1.8 | (n = 2) |
| 2 | 2-Me—C₆H₄ | 81.8 ± 0.7 | (n = 2) |
| 3 | 3,5-di-Me—C₆H₃ | 79.0 ± 1.5 | (n = 2) |
| 4 | 2,6-di-Me—C₆H₃ | 28.1 ± 6.1 | (n = 3) |
| 5 | 2-MeO—C₆H₄ | 12.0 ± 1.2 | (n = 2) |
| 6 | 4-MeO—C₆H₄ | 95.1 ± 0.5 | (n = 2) |
| 7 | 3,4-di-MeO—C₆H₃ | 98.1 ± 0.8 | (n = 2) |
| 8 | 2-CF₃—C₆H₄ | 77.8 ± 0.9 | (n = 2) |
| 9 | 2-Br—C₆H₄ | 14.7 ± 2.8 | (n = 2) |
| 10 | 2-CN—C₆H₄ | 71.1 ± 8.7 | (n = 3) |
| 11 | 3-NO₂—C₆H₄ | 95.0 ± 1.3 | (n = 2) |
| 12 | 3-CN—C₆H₄ | 86.1 ± 7.7 | (n = 3) |
| 13 | 4-NHAc—C₆H₄ | 93.6 ± 1.4 | (n = 2) |
| 14^{a} | 4-OH—C₆H₄ | 86.7 ± 4.6 | (n = 3) |
| 15 | 2-OH,4-NO₂—C₆H₃ | 15.2 ± 2.0 | (n = 2) |
| 16 | 4-I—C₆H₄ | 93.5 ± 1.2 | (n = 2) |
| 17 | 4-CO₂Et—C₆H₄ | 94.7 ± 0.4 | (n = 2) |
| 18 | 4-CF₃—CC₆H₄ | 95.3 ± 1.3 | (n = 2) |
| 19^{a} | 4-CO₂H—C₆H₄ | 94.2 ± 1.8 | (n = 2) |
| 20 | 4-NO₂—C₆H₄ | 88.2 ± 9.9 | (n = 3) |
| 21 | 2-furanyl | 93.8 ± 0.2 | (n = 2) |
| 22 | 2-chloropyridin-4-yl | 93.8 ± 0.3 | (n = 2) |
| 23 | 2-bromopyridin-3-yl | 92.9 ± 2.3 | (n = 2) |
| 24 | 2-chloropyridin-3-yl | 89.9 ± 2.0 | (n = 2) |
| 25 | benzo[b]thiophen-2-yl | 88.9 ± 8.8 | (n = 3) |
| 26 | 4-CH₂Br—C₆H₄ | 20 ± 4 | (n = 3) |
| 27 | 4-CHO—C₆H₄ | 92 | (n = 1) |
| 28 | 4-NH₂—C₆H₄ | 91 ± 4 | (n = 3) |

^{a}These reactions were performed in the absence of Et₃•3HF.

### Example 12. Preparation of [¹¹C](Trifluoromethyl)Benzene from Phenyl(2,4,6-Trimethoxyphenyl)Iodonium Tosylate

The general procedure for reaction of [¹¹C]CuCF₃ with diaryliodonium salts when applied to phenyl(2,4,6-trimethoxyphenyl)iodonium tosylate gave [¹¹C](trifluoromethyl)benzene in 99% yield. This example shows that the 2,4,6-trimethoxyphenyl group is an effective spectator group in these reactions.

### Example 13. Preparation of Substituted Styryl [¹¹C]Trifluoromethylarenes

Table 6 shows the yields of substituted [¹¹C]trifluoromethylarenes obtained from phenyl(vinyl)iodonium tosylates and [¹¹C]CuCF₃ performed under the reaction conditions of Example 10, Table 4, entry 5 (phenyl(vinyl)iodonium salt (50 μmol); DMF (100-200 μL); [¹¹C]CuCF₃ in DMF (approximately 0.2 mL); 10 min; RT). These are non-limiting examples exemplifying the scope of the reaction.

TABLE 6
Yields from the reactions of phenyl(vinyl)iodonium tosylates with
[¹¹C]CuCF₃.

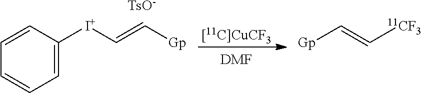

| Entry | Gp | Yield of Gp—¹¹CF₃ (%) |
|---|---|---|
| 1 | Ph | 97 (n =1) |
| 2 | 4-Me—C₆H₄ | 98 (n =1) |
| 3 | 4-MeO—C₆H₃ | 96 (n =1) |
| 4 | 4-F—C₆H₃ | 96 (n =1) |
| 5 | 4-Ph—C₆H₄ | 86 (n =1) |
| 6 | 4-CF₃—C₆H₄ | 98 (n =1) |
| 7 | 2-F—C₆H₄ | 94 (n =1) |
| 8 | 2-Cl—C₆H₄ | 92 (n =1) |
| 9 | 1-naphthalenyl | 98 (n =1) |
| 10 | cyclohexyl | 96 (n =1) |

### Example 14: Use of [¹¹C]CuCF₃ to Label Drug Examples in Trifluoromethyl Groups

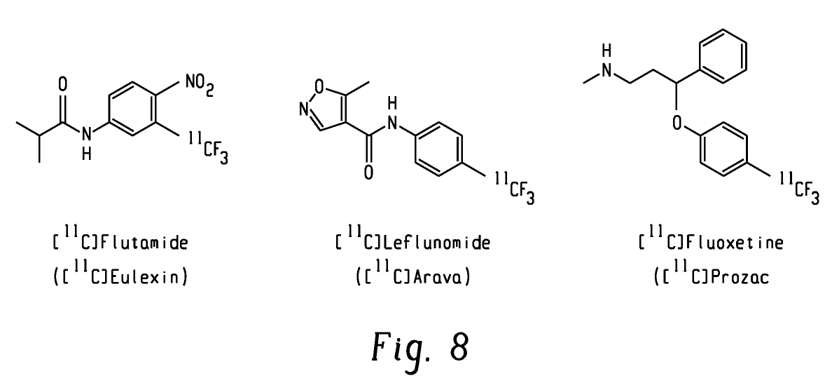

The availability of [¹¹C]fluoroform in high molar activity opened up the possibility to label trifluoromethyl groups with carbon-11 in drug-like or PET radiotracer-type molecules. To exemplify this possibility, [¹¹C]CuCF₃ was used to label three known drugs, namely the antiandrogen flutamide (Eulexin®), the antirheumatic drug leflunomide (Arava®), and the antidepressant fluoxetine (Prozac®) (FIG. 8).

[¹¹C]Flutamide was obtained in 76±8% yield (n=3) from the corresponding iodo-precursor.

[¹¹C]Leflunomide was labeled in 93±5% yield (n=3) from the corresponding boronic acid precursor, and with a high molar activity of 400 GBq/μmol (corrected to end of radionuclide production).

[¹¹C]Leflunomide was obtained in 86±2% yield (n=2) from the reaction of [¹¹C]CuCF₃ on mesityl(4-(5-methylisoxazole-4-carboxamido)phenyl)iodonium tosylate, with a high molar activity of 642 GBq/μmol (n=1) (corrected to end of radionuclide production).

[¹¹C]Fluoxetine was obtained in 45±4% yield (n=3) from the reaction of [¹¹C]CuCF₃ on a Boc-protected iodo precursor, followed by deprotection.

[¹¹C]Fluoxetine was obtained in 82±2% yield (n=2) from the reaction of [¹¹C]CuCF₃ on mesityl(4-(3-(methylamino)-1-phenylpropoxy)phenyl)iodonium tosylate.

The predominantly high yields in the vast majority of the above reaction examples with [¹¹C]fluoroform or [¹¹C]CuCF₃ attest to the absence of troublesome impurities in the collected [¹¹C]fluoroform; only low levels of minor unreactive fluorinated-¹¹C species were ever seen as contaminants.

### Example 15. Preparation of [¹¹C]2,2,2-Trifluoro-1-Phenylethanone by Reaction of [¹¹C]Fluoroform with Methyl Benzoate

Methyl benzoate (7 mg) and t-BuOK (5 mg) were added to a 1 mL glass vial with DMF (0.4 mL) and capped. [¹¹C]Fluoroform (100-300 μL) was added. The mixture was left to react at RT for 10 min, and added 0.1 mL of HCl (37%) and reacted at 60° C. for 5 min. The reaction mixture was diluted with MeCN:H₂O (3:1, v/v), and analyzed with HPLC. [¹¹C]2,2,2-trifluoro-1-phenylethanenon was obtained in 65±17% yield (n=3).

### Example 16. Preparation of [¹⁸F]Fluoromethane ([¹⁸F]CH₃F) from Cyclotron Produced [¹⁸F]Fluoride Ion and Applications

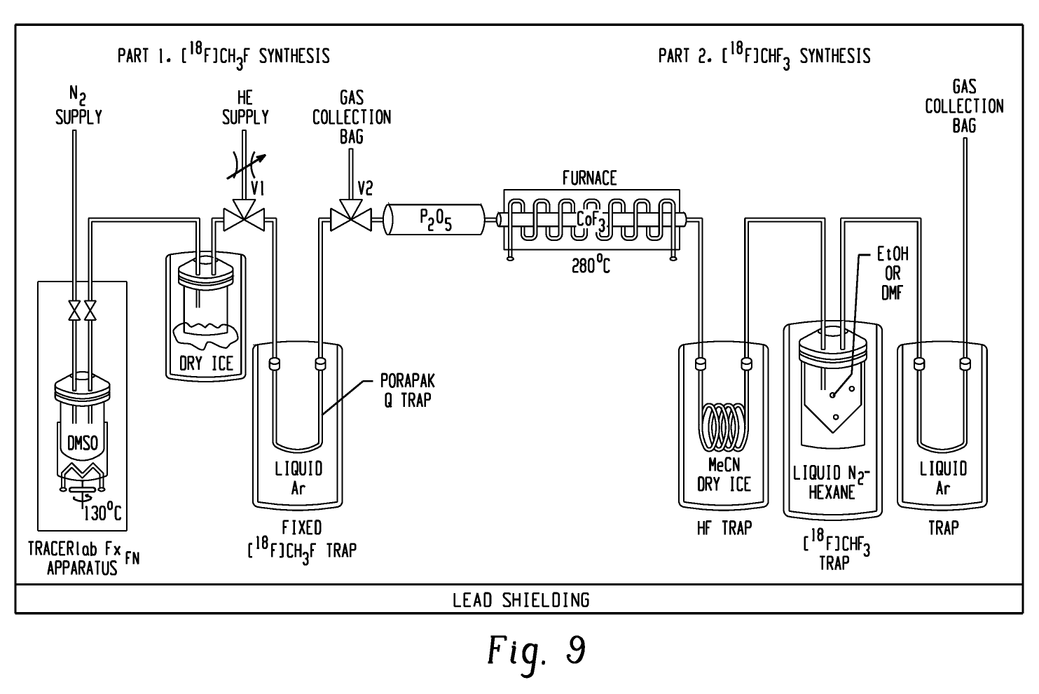

The procedure was performed in a TracerLab FxFN (GE) module (FIG. 9). Proton irradiated [¹⁸O]water from a cyclotron containing [¹⁸F]fluoride ion (<37 GBq), K₂CO₃ (20 μmol)/K 2.2.2 (kryptofix 2.2.2; 20 μmol) stock solution (100 μL) was loaded into a glass vial. Acetonitrile (2 mL) was added and the solvent was azeotropically removed at 88° C. under a stream of nitrogen gas vented to vacuum. This drying process was repeated twice. A solution of methyl methanesulfonate (MeOMs), methyl triflate (MeOTf), or methyl nosylate (MeONs) (8.5 μL, 0.1 mmol) in DMSO (1 mL) was transferred to the drying vial at 60° C. The reaction mixture was allowed to stand at 100° C. for 30 min. The reaction vial was kept closed until it had cooled down to 35° C. The generated [¹⁸F]fluoromethane was flushed out of the reaction vial with nitrogen gas at 20 mL/min and trapped on PORAPAK™ Q (1 g) in a U-shaped stainless steel tube cooled in liquid argon (−186° C.). The dependence of [¹⁸F]fluoromethane synthesis yield (%) on selection of precursor is shown in Table 7.

TABLE 7
Dependence of [¹⁸F]fluoromethane
yield (%) on selection of precursor

| | | | Reaction | Reaction | Recovered |
|---|---|---|---|---|---|
| | b.p | K⁺/K | Temp. | Time | yield |
| Precursor | (° C.) | 2.2.2 | (° C.) | (min) | (%) |
| MeONs | 89-92 | 0.54 | 100 | 30 | 0.28 |
| MeOTf | 94-99 | 0.54 | 100 | 30 | 0.08 |
| MeOMs | 202-203 | 0.54 | 100 | 30 | 59.37 |

Methyl methanesulfonate was selected as the precursor for [¹⁸F]fluoromethane synthesis in subsequent examples.

Temperature Dependence of [¹⁸F]Fluoromethane Synthesis.

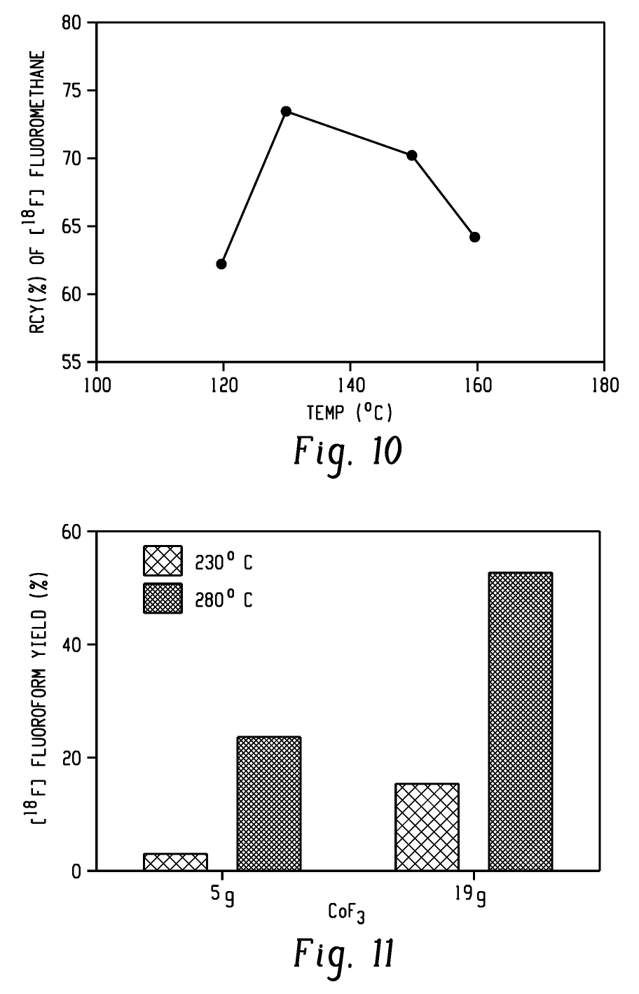

After selection of a precursor for [¹⁸F]fluoride ion methylation, [¹⁸F]fluoromethane was synthesized at several temperatures. Synthesis methods were as described earlier for preparation of [¹⁸F]fluoromethane. A high yield of [¹⁸F]fluoromethane was obtained at 130° C. (FIG. 10). The average yield of [¹⁸F]fluoromethane in the 120 to 160° C. temperature range was 68±5% (n=1).

Calculation of Conversion of [¹⁸F]Fluoride Ion into [¹⁸F]Fluoromethane.

The percentage conversion of [¹⁸F]fluoride ion into [¹⁸F]fluoromethane was calculated as the [¹⁸F]fluoromethane trapped in the PORAPAK™ Q trap before release, divided by the radioactivity of [¹⁸F]fluoride ion, and multiplied by 100. The yield of [¹⁸F]fluoromethane was 49±14% (n=48) (corrected to the end of radionuclide production).

[¹⁸F]Fluoroform Synthesis.

The synthesis of [¹⁸F]Fluoroform was performed in an in-house built apparatus (FIG. 9) similar to the apparatus in FIG. 1. [¹⁸F]Fluoromethane was released from the U-shaped stainless steel tube by allowing it to warm to RT. Subsequently, a controlled flow of helium gas (20 mL/min) was used to carry the [¹⁸F]fluoromethane out of the tube and over phosphorus pentoxide (Sicapent®) packed into a stainless steel column, and then over a heated (280° C.) CoF₃ (19 g) column. The generated [¹⁸F]fluoroform was passed through a trap cooled at approximately −40° C. to trap HF and finally trapped in a glass V-vial containing DMF (0.6-1.0 mL) and cooled at approximately −40° C. Another U-shaped stainless steel tube containing PORAPAK™ Q (1 g) was connected to the outlet of the V-shaped glass product vial to measure any breakthrough of radioactive material. The yield of [¹⁸F]fluoroform was 41±6% (n=30).

Calculation of Recovery Yield of [¹⁸F]Fluoromethane Ion into [¹¹F]Fluoroform.

The percentage conversion of [¹⁸F]fluoromethane into [¹⁸F]fluoroform was calculated as the [¹⁸F]fluoromethane trapped in the cold DMF, divided by radioactivity of [¹⁸F]fluoromethane, and multiplied by 100 (FIG. 10). All radioactivities were decay corrected to end of radionuclide production.

CoF₃ Amount and Temperature Dependence of [¹⁸F]Fluoroform Synthesis.

Two columns of CoF₃, one containing 5 g and another 19 g, were tested for the synthesis of [¹⁸F]fluoroform. High yields of [¹⁸F]fluoroform were obtained in fluorination when using 19 g of CoF₃ as a packed column at 280° C. (FIG. 11).

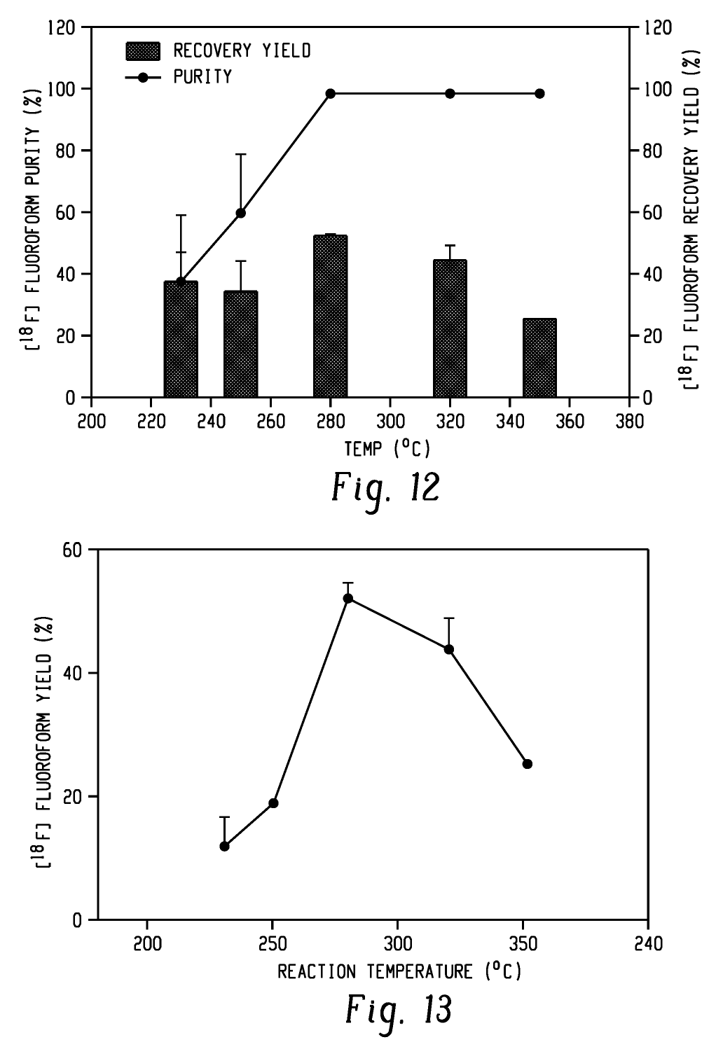

High yields of [¹⁸F]fluoroform were obtained between 280 and 320° C., with 280° C. appearing optimal (FIG. 12). The average yield of [¹⁸F]fluoroform in the 230 to 350° C. temperature range was 40±10%, based on 9 experiments conducted with single CoF₃ column.

Yield Calculation for [¹⁸F]Fluoroform.

The percentage of radioactivity represented by [¹⁸F]fluoroform in the HPLC analyte was calculated from the radio-HPLC chromatogram from peak areas with a small correction for radioactive decay between radioactive peaks during the analysis. Radioactivity trapped in the cold DMF was found to be the major portion of radioactivity that had been introduced initially into the apparatus. Therefore, the yield of [¹⁸F]fluoroform was calculated as the percentage of radioactivity represented by [¹⁸F]fluoroform in the HPLC analyte multiplied by the percentage recovery of initial radioactivity recovered in the cold DMF (FIG. 13).

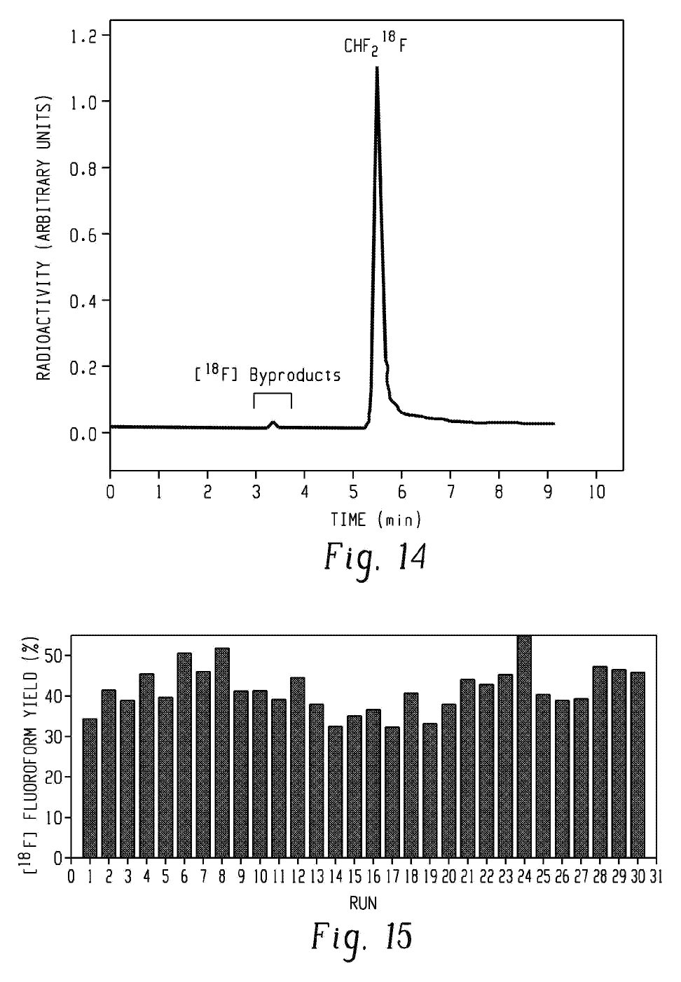

FIG. 14 shows the HPLC analysis of trapped [¹⁸F]fluoroform. HPLC conditions were as described earlier for the analysis of [¹¹C]fluoroform.

Repeatability of CoF₃ Column Performance.

FIG. 15 shows the yield of [¹⁸F]fluoroform with respect to the number of CoF₃ column uses. The yields of [¹⁸F]fluoroform were reliable over 30 runs (41±6%).

Conversion of [¹⁸F]fluoroform into [¹⁸F]CuCF₃. Within a glovebox (having a dry nitrogen atmosphere), t-BuOK in DMF (0.3 M, 50 μL) was added to CuBr (0.7 mg, 5 μmol) in a dry 1-mL glass vial. The vial was septum-sealed and removed from the glovebox. [¹⁸F]Fluoroform in DMF (50-300 μL) was added to the vial, mixed, and left at RT for 1 min. A solution of Et₃N.3HF in DMF (1.64% v/v; 50 μL) was then added. The mixture was mixed thoroughly and held at RT for another minute, before use in labeling reactions.

### Example 17: Use of [¹⁸F]Fluoroform ([¹⁸F]CHF₃) and the Derivative [¹⁸F]CuCF₃ for Compound Labeling

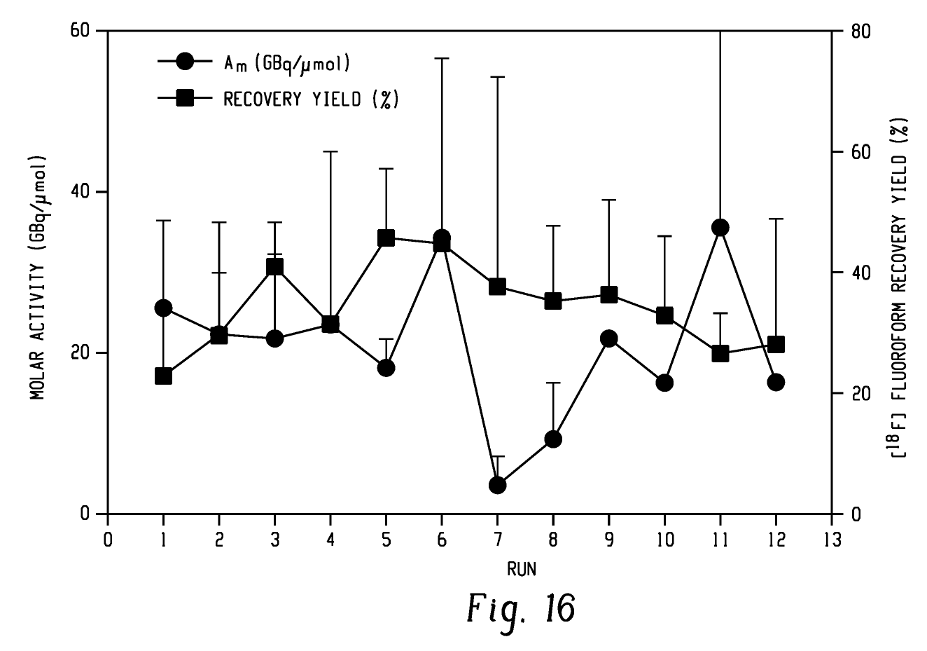

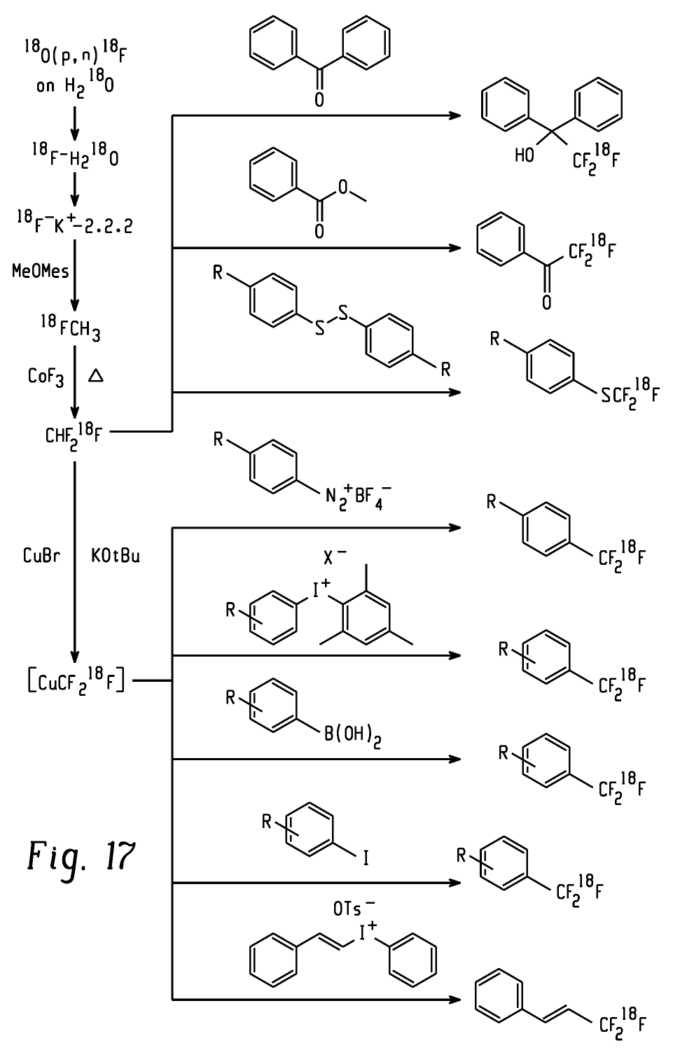

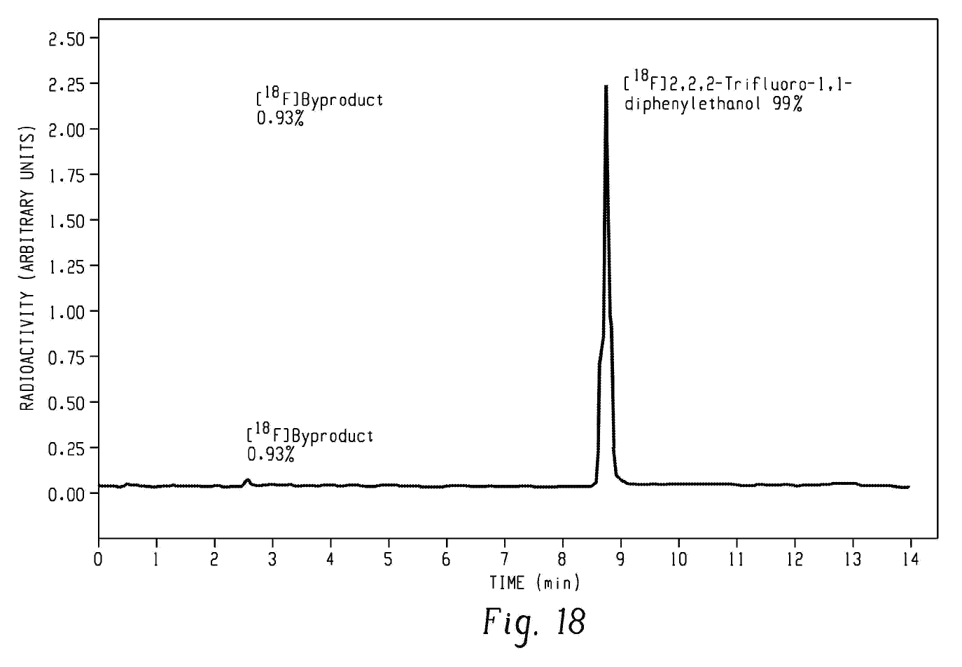

The following non-limiting examples show that [¹⁸F]fluoroform and [¹¹C]fluoroform prepared by the processes described herein have the same chemical reactivity. FIG. 16 exemplifies radiochemistry performed with [¹⁸F]fluoroform or [¹⁸F]CuCF₃.

Preparation of [¹⁸F]trifluoromethylarene from [¹⁸F]fluoroform and a diaryl Disulfane ((ArS)₂; Referred to Generally Herein as a Diaryl Disulfide).

Within a glovebox, freshly prepared t-BuOK (0.3 M) in DMF (50 μL) and CsF (6 mg; 40 μmol) were septum-sealed within a dry 1-mL glass vial. [¹⁸F]Fluoroform in DMF (50-200 μL) was then added. A diaryl disulfane (100 μmol) in DMF (0.1 mL) was then added and the mixture heated to 100° C. for 10 min.

Preparation of a [¹⁸F]Trifluoromethylarene from [¹⁸F]CuCF₃ and an Aryl Diazonium Salt.

A solution of diazonium salt (50 μmol) in DMF (100 μL) was added to freshly prepared [¹⁸F]CuCF₃ in DMF (approximately 0.2 mL) within a dry septum-sealed glass vial and held for 10 min at RT.

Preparation of a [¹⁸F]Trifluoromethylarene from [¹⁸F]CuCF₃ and a Diaryliodonium Salt.

A solution of diaryliodonium salt (50 μmol) in DMF (100 μL) was added to [¹⁸F]CuCF₃ in DMF (approximately 0.2 mL) within a dry septum-sealed glass vial and held for 10 min at RT.

Preparation of a [¹⁸F]Trifluoromethylarene from [¹⁸F]CuCF₃ and an Arylboronic Acid.

A solution of an arylboronic acid (50 μmol) in DMF (100 μL) was added to [¹⁸F]CuCF₃ in DMF (approximately 0.2 mL) within a dry septum-sealed glass vial. Air (10 mL) was then bubbled through the mixture at RT.

Preparation of a [¹⁸F]Trifluoromethylarene from [¹⁸F]CuCF₃ and an Aryl Iodide.

A solution of aryl iodide (100 μmol) in DMF (100 μL) was added to [¹⁸F]CuCF₃ in DMF (approximately 0.2 mL) within a dry septum-sealed glass vial, heated to 130° C. for 5 min, and then cooled to RT

### Example 18. Specific Examples of the Application of [¹⁸F]Fluoroform to Labeling Model Compounds in Trifluoromethyl Groups

Labeled Product Analysis.

Radioactive products from reactions of [¹⁸F]fluoroform or [¹⁸F]CuCF₃ (except those from reaction with leflunomide, 1,3-dimethyl-5-(trifluoromethyl)pyrimidine-2,4(1H,3H)-dione, 2,6-di-tert-butoxy-1,2,3,4-tetrahydropyrimidin-5-ylboronic acid) were quenched with MeCN-0.1% TFA H₂O (3:1 v/v; 1-3 mL), filtered through a syringe filter (0.2 μm, PTFE) and analyzed with reversed phase HPLC on a Luna® C18 column (10 μm; 250×4.6 mm). Products were eluted at 2 mL/min with a gradient of 0.1% TFA H₂O (A)-MeCN (B), with A started at 55% (v/v) for 1 min, increased linearly to 80% over 10 min, and held at 80% for 5 min.

Labeled products from benzophenone were analyzed with HPLC on a column (Luna®, 10 μm, 250×4.6 mm). Products were eluted at 2 mL/min with a mobile phase gradient of H₂O (A)-MeCN (B) with A started at 45% for 1 min, increased linearly to 80% over 10 min, and held at 80% for another 5 min.

Labeled products from leflunomide, 1,3-dimethyl-5-(trifluoromethyl)pyrimidine-2,4(1H,3H)-dione, 2,6-di-tert-butoxy-1,2,3,4-tetrahydropyrimidin-5-ylboronic acid, were quenched with 0.1% TFA-MeCN (1:1, 1-3 mL), filtered through a syringe filter (0.2 μm, PTFE) and analyzed with HPLC on a column (Luna®, 10 μm, 250×4.6 mm). Products were eluted at 2 mL/min with a mobile phase gradient of 0.1% TFA H₂O (A)-MeCN (B) with A started at 10% for 1 min, increased linearly to 80% over 15 min and kept at 80% for another 5 min.

Eluates were monitored for absorbance at 254 nm and radioactivity with a scintillation detector (Bioscan; Washington D.C.). The identities of labeled products were confirmed by their comobilities with authentic reference compounds.

Molar Activity Determination.

The radio-HPLC system for analysis of [¹⁸F]fluoroform reaction products was calibrated for UV absorption response versus mass of reference compound in the analyte. This allowed the mass of carrier in the injectate to be calculated and the molar activity to be calculated from the additional measurement of radioactivity in the labeled compound peak. The molar activity was decay-corrected to the end of radionuclide production.

### Example 19. Synthesis of [¹⁸F]2,2,2-trifluoro-1,1-diphenylethanol

### Synthesis of [¹⁸F]2,2,2-trifluoro-1,1-diphenylethanol by reaction of [¹⁸F]fluoroform with benzophenone

Benzophenone (9-13 mg) and t-BuOK (5 mg) were added to a 1-mL glass vial with DMF (0.4 mL) and capped. [¹⁸F]Fluoroform (100-300 μL) was added. The mixture was left to react at RT for 10 min, diluted with MeCN:H₂O (3:1, v/v), and analyzed with HPLC. [¹⁸F]2,2,2-Trifluoro-1,1-diphenylethanol was produced in quantitative yield (n=5). The molar activity in one preparation was measured to be 54.29 GBq/μmol. FIG. 14 shows the radiochromatogram from the HPLC analysis of the reaction mixture.

Molar Activity Measurement with [¹⁸F]2,2,2-Trifluoro-1,1-Diphenylethanol and Comparison with Conventional ¹⁸F-Fluorination Methods.

Molar activity measurement from [¹⁸F]fluoroform labeling was compared with a conventional ¹⁸F-fluorination method. Measurement was performed at same day with same batch of cyclotron produced [¹⁸F]fluoride ion in H₂180 water. As shown in Table 8, the molar activities from [¹⁸F]fluoroform labeling were about 9 times diluted compare to a conventional ¹⁸F-fluorination method.

TABLE 8
Molar activity comparison between [¹⁸F]2,2,2-
trifluoro-1,1-diphenylethanol and conventionally prepared
¹⁸F-fluorinated compounds.

| | [¹⁸F]fluoroform | Conventional |
|---|---|---|
| | labeling | ¹⁸F-Fluorination |
| Entry | (GBq/μmol) | (GBq/μmol) |
| 1 | 22 | 305 |
| 2 | 17 | 235 |
| 3 | 54 | 573 |
| 4 | 17 | 158 |
| 5 | 42 | 120 |
| Average | 30 ± 17 | 278 ± 180 |

Regeneration of CoF₃ Column.

The repeatability of the CoF₃ column for synthesis of [¹⁸F]fluoroform is shown in FIG. 15. Molar activity steadily decreased with use Regeneration was performed by heating the CoF₃ column at 280° C. with a continuous He(g) flow (20 mL/min) for several hours. The results are provided in FIG. 16 and Table 9. The molar activity recovered after each re-baking.

TABLE 9
Regeneration of CoF₃ column.

| | | | | |
|---|---|---|---|---|
| | Temp | He(g) | Time | Molar activity |
| Entry | (° C.) | (20 mL/min) | (h) | (GBq/ μmol) |

Column 1

| | | | | |
|---|---|---|---|---|
| 0 | N/A | N/A | N/A | 5 |
| 1 | 280 | continuous | 3 | 22 |
| 2 | 280 | continuous | 3 | 17 |
| 3 | 280 | continuous | 15 | 54 |
| 4 | 280 | continuous | 8 | 17 |

Column 2

| | | | | |
|---|---|---|---|---|
| 0 | 280 | continuous | 20 | 29 |
| 1 | 280 | continuous | 20 | 23 |
| 2 | 280 | continuous | 20 | 27 |
| 3 | 280 | continuous | 20 | 0 |
| 4 | 280 | continuous | 20 | 15 |
| 5 | 280 | continuous | 20 | 43 |
| 6 | 280 | continuous | 20 | 1 |
| 7 | 280 | continuous | 20 | 14 |
| 8 | 280 | continuous | 20 | 21 |
| 9 | 280 | continuous | 20 | 16 |
| 10 | 280 | continuous | 20 | 17 |
| 11 | 280 | continuous | 20 | 16 |
| 12 | 280 | continuous | 20 | 13 |
| 13 | 280 | continuous | 20 | 0 |
| 14 | 280 | continuous | 20 | 0 |

Column 3

| | | | | |
|---|---|---|---|---|
| 0 | 280 | continuous | 20 | 14 |
| 1 | 280 | continuous | 20 | 16 |
| 2 | 280 | continuous | 20 | 10 |
| 3 | 280 | continuous | 20 | 42 |
| 4 | 280 | continuous | 20 | 18 |
| 5 | 280 | continuous | 20 | 52 |

### Example 20. Synthesis of [¹⁸F]p-tolyl(trifluoromethyl)sulfane from 1,2-di-p-tolyldisulfane and [¹⁸F]fluoroform

Treatment of 1,2-Di-p-tolyldisulfane with [¹⁸F]CuCF₃ gave [¹⁸F]p-tolyl(trifluoromethyl)sulfane in 15.9% yield (n=1).

### Example 21. Synthesis of [¹⁸F]4-(trifluoromethylthio)benzonitrile from 4,4′-disulfanediyldibenzonitrile and [¹⁸F]fluoroform

Treatment of 4,4′-disulfanediyldibenzonitrile with [¹⁸F]CuCF₃ gave [¹⁸F]4-(trifluoromethylthio)benzonitrile in 61.3% yield (n=1).

### Example 22. Preparation of [¹⁸F](trifluoromethyl)arenes from [¹⁸F]CuCF₃ and aryldiazonium salts

The earlier general methods described for reaction of [¹⁸F]CuCF₃ with aryldiazonium salts gave the results summarized in Table 10.

TABLE 10
Yields of [¹⁸F]trifluoromethylarenes from reactions of aryldiazonium
salts with [¹⁸F]CuCF₃.

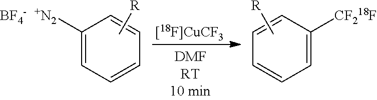

| Entry | R | Yield of ¹⁸F-labeled product (%) |
|---|---|---|
| 1 | 4-NO₂ | 81.1 ± 4.6 (n = 3) |
| 2 | 4-MeO | 96.3 ± 0.5 (n = 3) |
| 3 | 4-Br | 80.0 ± 12.6 (n = 2) |
| 4 | 3,5-di-Cl | 39.4 ± 17.5 (n = 2) |

### Example 23. Syntheses of [¹⁸F]trifluoromethylarenes from mesityl(aryl)iodonium salts and [¹⁸F]fluoroform

The earlier general methods described for reaction of [¹⁸F]CuCF₃ with aryliodonium salts gave the results summarized in Table 11. These are comparable to the yields from similar reactions with [¹¹C]fluoroform.

TABLE 11
Syntheses of [¹⁸F]trifluoromethylarenes from mesityl(aryl)iodonium
salts and [¹⁸F]fluoroform

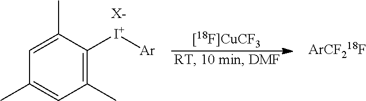

| Entry | Ar | Yield of ArCF₂¹⁸F (%) | |
|---|---|---|---|
| 1 | Ph | 78.1 ± 11.7 | (n = 2) |
| 2 | 2-Me—C₆H₄ | 78.0 ± 6.8 | (n = 3) |
| 3 | 4-MeO—C₆H₄ | 77.2 ± 15.5 | (n = 2) |
| 4 | 3,4-di-MeO—C₆H₃ | 98.8 ± 1.5 | (n = 3) |
| 5 | 3-F—C₆H₄ | 90.3 ± 10.0 | (n = 3) |
| 6 | 3-CF₃—C₆H₄ | 85.8 ± 2.2 | (n = 2) |
| 7 | 3-NO₂—C₆H₄ | 70.7 ± 19.9 | (n = 3) |
| 8 | 4-CN—C₆H₄ | 84.2 ± 0.5 | (n = 2) |
| 9 | 4-OH—C₆H₄ | 90.9 ± 7.0 | (n = 2) |
| 10 | 4-I—C₆H₄ | 87.0 ± 2.4 | (n = 3) |
| 11 | 4-CO₂Et—C₆H₄ | 95.3 ± 5.7 | (n = 2) |
| 12 | 4-CO₂H—C₆H₄ | 94.6 ± 2.9 | (n = 3) |
| 13 | 4-NO₂—C₆H₄ | 88.7 ± 5.1 | (n = 2) |
| 14 | 4-NH₂—C₆H₄ | 93.4 ± 0.9 | (n = 2) |
| 15 | Furan-2-y1 | 98.2 ± 1.2 | (n = 2) |
| 16 | benzo[b]thiophen-2-y1 | 96.3 ± 5.0 | (n = 3) |

^{a} This is mesityl(aryl)iodonium triflate. All others are mesityl(aryl)iodonium tosylates.

### Example 24. Preparation of [¹⁸F](Trifluoromethyl)Arenes from [¹⁸F]CuCF₃ and Boronic Acids

The earlier general methods described for reaction of [¹⁸F]CuCF₃ with boronic acids gave the results summarized in Table 12.

TABLE 12
Syntheses of [¹⁸F]trifluoromethylarenes from boronic acids and
[¹⁸F]fluoroform

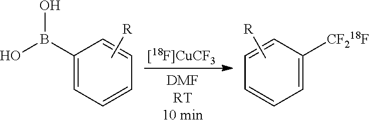

| Entry | R | Yield of ¹⁸F-labeled product (%) (n = 3) |
|---|---|---|
| 1 | 2-MeO | 95.9 ± 3.3 |
| 2 | 2,4,6-Trimethylphenyl | 90.4 ± 2.9 |
| 3 | 4-formylphenyl | 99.2 ± 1.1 |
| 4 | 4-(Bromomethyl)phenyl | 7.1 ± 3.1 |
| 5^{a} | 2,4-Di(tert- | 95.9 ± 3.6 |
| | butoxy)pyrimidine | |

^{a}tert-butoxide groups were deprotonated using 0.2 mL of HCl (37%) at 80° C. for 2 min.

### Example 25. Preparation of [¹⁸F]Leflunomide with [¹⁸F]CuCF₃ and Corresponding Iodophenyl Precursor

Treatment of N-(4-iodophenyl)-5-methylisoxazole-4-carboxamide with [¹⁸F]CuCF₃ gave [¹⁸F]leflunomide in 43.9±4.1% yield (n=3).

### Example 26. Preparation of [¹⁸F]1,3-dimethyl-5-(trifluoromethyl)pyrimidine-2,4(1H,3H)-dione with [¹⁸F]CuCF₃ and 5-iodo-1,3-dimethylpyrimidine-2,4(1H,3H)-dione

Treatment of N-(4-iodophenyl)-5-methylisoxazole-4-carboxamide with [¹⁸F]CuCF₃ gave [¹⁸F]1,3-dimethyl-5-(trifluoromethyl)pyrimidine-2,4(1H,3H)-dione in 98.0±3.4% yield (n=3).

### Example 27. Preparation of [¹⁸F]4-trifluoromethyl-L-phenylalanine with [¹⁸F]CuCF₃ and (S)-methyl-2-boc-amino-3-(4-iodophenyl)propionate

Treatment of (S)-methyl-2-boc-amino-3-(4-iodophenyl)propionate with [¹⁸F]CuCF₃ and added 0.1 mL of HCl (37%) and reacted at 130-C for 5 min. [¹⁸F]4-trifluoromethyl-L-phenylalanine was obtained in 68±6% yield (n=3).

### Example 28. Preparation of [¹⁸F]2,2,2-trifluoro-1-phenylethanone by Reaction of [¹⁸F]fluoroform with Methyl Benzoate

Methyl benzoate (7 mg) and t-BuOK (5 mg) were added to a 1 mL glass vial with DMF (0.4 mL) and capped. [¹⁸F]Fluoroform (100-300 μL) was added. The mixture was left to react at RT for 10 min, and added 0.1 mL of HCl (37%) and reacted at 60° C. for 5 min. The reaction mixture was diluted with MeCN:H₂O (3:1, v/v), and analyzed with HPLC. [¹⁸F]2,2,2-trifluoro-1-phenylethanone was obtained in 75±7% yield (n=3).

### Example 29

The general procedure for reaction of [¹⁸F]CuCF₃ with diaryliodonium salts when applied to (E)-phenyl(styryl)iodonium tosylate gave [¹⁸F](E)-(3,3,3-trifluoroprop-1-enyl)benzene in 99±1% yield (n=2).

### CONCLUSIONS

[¹¹C]Fluoroform is readily and simply produced in useful yield from cyclotron-produced [¹¹C]methane by passage over heated CoF₃. The method is rapid, robust, and low-maintenance. With this development, the labeling of PET radiotracers at trifluoromethyl groups in high molar activity sufficient for imaging low density targets in human subjects is now possible. An enhanced range of exciting radiotracers for PET applications can now be developed based on adapting the known rich chemistry of fluoroform to [¹¹C]fluoroform for previously unprecedented ¹¹C-labeling at carbon atoms bearing more than one fluorine atom

[¹⁸F]Fluoroform is readily and simply produced in useful yield from cyclotron-produced [¹⁸F]fluoromethane by passage over heated CoF₃. The method is rapid, robust, and low-maintenance. With this development, the labeling of PET radiotracers at trifluoromethyl groups in moderately high molar activity (comparable to the highest value so far reported by former methods) is now possible. An enhanced range of radiotracers for PET applications are available based on adapting the known rich chemistry of fluoroform to [¹⁸F]fluoroform for previously unprecedented ¹⁸F-labeling at carbon atoms bearing more than one fluorine atom.

Unprecedented methods are described in this invention for introducing carbon-11 at high molar activity or fluorine-18 at moderately high molar activity into labeled compounds, and especially at trifluoromethyl groups.

These methods include reactions of [¹¹C]fluoroform or [¹⁸F]fluoroform with ketones or disulfides, in some aspects diaryl ketones or diaryl disulfides.

These methods also include reactions of the fluoroform derivatives [¹¹C]CuCF₃ or [¹⁸F]CuCF₃ with arylboronic acids, aryl iodides, aryldiazonium salts, diaryliodonium salts or aryl(vinyl)iodonium salts. The aryl groups can contain one or more heteroatoms in the rings. Other functional groups can be present, for example halogen, nitro, cyano, alkyl sulfonyl, aryl sulfonyl, aldehyde, ester, amido, alkyl, alkoxy, alkenyl, alkenyloxy, cycloalkyl, cycloalkyoxy, monosulfide, aryl, or aryloxy

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

Unless defined otherwise, technical and scientific terms used herein have the same meaning as is commonly understood by one of skill in the art to which this invention belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

The term “alkyl” means a branched or straight chain, unsaturated aliphatic group, e.g., methyl, ethyl, n-propyl, i-propyl, n-butyl, s-butyl, t-butyl, n-pentyl, s-pentyl, and n- and s-hexyl. “Alkenyl” means a straight or branched chain, monovalent group having at least one carbon-carbon double bond (e.g., ethenyl (—HC═CH₂)). “Alkynyl” means a straight or branched chain, monovalent group having at least one carbon-carbon triple bond (e.g., ethynyl (—HC═CH)). “Alkoxy” means an alkyl group that is linked via an oxygen (i.e., alkyl-O—), for example methoxy, ethoxy, and sec-butyloxy groups. “Alkylene” means a straight or branched chain, saturated, divalent aliphatic hydrocarbon group (e.g., methylene (—CH₂—) or, propylene (—(CH₂)₃—)). “Cycloalkyl” means a divalent cyclic alkyl group, —CₙH₂ₙ₋ₓ, wherein x is the number of hydrogens replaced by cyclization(s). “Aryl” means an group containing at least one aromatic ring containing at least one carbon atom and optionally one or more heteroatoms (e.g., 1, 2, or 3 heteroatoms, wherein each heteroatom is each independently N, O, S, Si, or P). A non-aromatic ring can also be present. Exemplary aryl groups include phenyl, indanyl, naphthyl, furyl, benzofuranyl, oxazolyl, benzoxazolyl, isoxazolyl, benzisoxazolyl, pyrroyl, indolyl, pyridinyl, pyrmidinyl, thiophenyl, benzothiophenyl, thiazolyl, pyrazolyl, triazinyl, but are not limited thereto. “Amino” includes a group of the formula —NH₂ as well as primary and secondary amines. Halogen and the prefix “halo” means a group or compound including one more of a fluoro, chloro, bromo, or iodo substituent. A combination of different halo groups (e.g., bromo and fluoro), or only chloro groups can be present.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A labeling agent that is [¹¹C]fluoroform, [¹¹C]difluoromethane, [¹¹C]fluoromethyl iodide, [¹¹C]fluoromethyl bromide, [¹¹C]fluoromethyl chloride, [¹¹C]fluoromethyl trifluoromethansulfonate, [¹¹C]difluoromethyl iodide, [¹¹C]difluoromethyl bromide, [¹¹C]difluoromethyl chloride, [¹¹C]difluoromethyl trifluoromethansulfonate, [¹¹C]trifluoromethyl iodide, [¹¹C]trifluoromethyl bromide, [¹¹C]trifluoromethyl chloride, [¹¹C]trifluoromethyl trifluoromethansulfonate, [¹⁸F]fluoroform, [¹⁸F]difluoromethane, [¹⁸F]difluoromethyl bromide, or [¹⁸F]trifluoromethylbromide.

2. (canceled)

3. The labeling agent of claim 1, wherein the labeling agent contains [¹¹C] and is NCA with a molar activity greater than 200 GBq/μmol, wherein the molar activity is corrected to the end of radionuclide production, or wherein the labeling agent contains [¹⁸F] and is has moderate molar activity greater that is greater than 15 GBq/μmol, wherein the molar activity is corrected to the end of radionuclide production.

4. (canceled)

5. A gas phase solvent-free method for producing an ¹¹C- or ¹⁸F-labeled fluoroalkane, the method comprising
contacting [¹¹C]methane, [¹⁸F]fluoromethane, [¹⁸F]fluoromethyl bromide, [¹¹C]methyl iodide, [¹¹C]methyl bromide, [¹¹C]methyl chloride, or [¹¹C]methyl trifluoromethansulfonate, with CoF₃ at a temperature of 50 to 450° C. and
isolating the ¹¹C- or ¹⁸F-labeled fluoroalkane that is produced.

6. The method of claim 5, wherein
the precursor is [¹¹C]methane and the labeled fluoroalkane is [¹¹C]fluoroform,
the precursor is [¹⁸F]fluoromethane and the labeled fluoroalkane is [¹⁸F]fluoroform,
the precursor is [¹⁸F]fluoromethane and the labeled fluoroalkane is [¹⁸F]difluoromethane,
the precursor is [¹⁸F]fluoromethyl bromide and the labeled fluoroalkane is [¹⁸F]difluoromethyl bromide,
the precursor is [¹⁸F]fluoromethyl bromide and the labeled fluoroalkane is [¹⁸F]trifluoromethyl bromide,
the precursor is [¹¹C]methyl iodide and the labeled fluoroalkane is [¹¹C]fluoromethyl iodide,
the precursor is [¹¹C]methyl bromide and the labeled fluoroalkane is [¹¹C]fluoromethyl bromide,
the precursor is [¹¹C]methyl chloride and the labeled fluoroalkane is [¹¹C]fluoromethyl chloride,
the precursor is [¹¹C]methyl trifluoromethansulfonate and the labeled fluoroalkane is [¹¹C]fluoromethyl trifluoromethansulfonate,
the precursor is [¹¹C]methyl iodide and the labeled fluoroalkane is [¹¹C]difluoromethyl iodide,
the precursor is [¹¹C]methyl bromide and the labeled fluoroalkane is [¹¹C]difluoromethyl bromide,
the precursor is [¹¹C]methyl chloride and the labeled fluoroalkane is [¹¹C]difluoromethyl chloride,
the precursor is [¹¹C]methyl trifluoromethansulfonate and the labeled fluoroalkane is [¹¹C]difluoromethyl trifluoromethansulfonate,
the precursor is [¹¹C]methyl iodide and the fluoroalkane is [¹¹C]trifluoromethyl iodide,
the precursor is [¹¹C]methyl bromide and the labeled fluoroalkane is [¹¹C]trifluoromethyl bromide,
the precursor is [¹¹C]methyl chloride and the labeled fluoroalkane is [¹¹C]trifluoromethyl chloride, or
the precursor is [¹¹C]methyl trifluoromethansulfonate and the labeled fluoroalkane is [¹¹C]trifluoromethyl trifluoromethansulfonate.

7. The method of claim 5, wherein the precursor contains less than 3 μmol of unlabeled carrier alkane.

8. The method of claim 5, wherein contacting does not include the addition of unlabeled carrier alkane.

9. The method of claim 5, further comprising removing impurities from the precursor prior to the contacting with CoF₃.

10. The method of claim 5, further comprising removing water and/or ammonia from the precursor prior to the contacting with CoF₃.

11. The method of claim 5, wherein the contacting is done in a flow of helium gas.

12. The method of claim 5, wherein the ¹¹C- or ¹⁸F-labeled fluoroalkane that is produced is trapped in a cold trap.

13. The method of claim 5, wherein the ¹¹C-labeled fluoroalkane that is produced is NCA with a molar activity greater than 200 GBq/μmol, wherein the molar activity is corrected to the end of radionuclide production.

14. The method of claim 5, wherein the radioactive precursor is either [¹¹C]methane or [¹⁸F]fluoromethane, and the radioactive precursor is contacted with the CoF₃ at a temperature of 260 to 290° C.

15. A method of preparing an ¹¹C-labeled or ¹⁸F-labeled radiotracer, comprising
combining [¹¹C]fluoroform or [¹⁸F]fluoroform with a non-radioactive precursor to form a reaction mixture, and
producing the ¹¹C-labeled radiotracer or the ¹⁸F-labeled radiotracer from the reaction mixture,
wherein the non-radioactive precursor contains a functionality that is reactive with the [¹¹C]fluoroform or the [¹⁸F]fluoroform,
or
converting [¹¹C]fluoroform or [¹⁸F]fluoroform into [¹¹C]CuCF₃ or [¹⁸F]CuCF₃,
combining the [¹¹C]CuCF₃ or [¹⁸F]CuCF₃ with a non-radioactive precursor to form a reaction mixture, and
producing the ¹¹C-labeled radiotracer or the ¹⁸F-labeled radiotracer from the reaction mixture,
wherein the non-radioactive precursor contains a functionality that is reactive with the [¹¹C]CuCF₃ or [¹⁸F]CuCF₃.

16. (canceled)

17. The method of claim 15, wherein the non-radioactive precursor is a diaryl ketone, a diaryl disulfide, an arylcarboxylic ester, an arylboronic acid, an aryl iodide, an aryldiazonium salt, a diaryliodonium salt, or an aryl(vinyl)iodonium salt.

18. An apparatus for gas phase production of a radiolabeled [¹¹C]- or [¹⁸F]-fluoroalkane from a ¹¹C- or ¹⁸F-labeled precursor, comprising,
an inlet for radiolabeled precursor in fluid communication with an impurity trap for removal of impurities from the radiolabeled precursor,
the impurity trap in fluid communication with a column for removal of ammonia and/or water from the radiolabeled precursor,
the column in fluid communication with a furnace suitable for heating CoF₃ to a temperature of 50 to 450° C.,
the furnace optionally in fluid communication with a hydrogen fluoride trap, and the furnace or the hydrogen fluoride trap in fluid communication with a product trap.

19. A method of imaging an organ or an area of a subject, comprising
administering to the subject a detectable amount of the radiotracer of claim 15,
imaging the organ or area of the subject using a nuclear medicine molecular imaging technique.

20. The method of claim 19, wherein the nuclear medicine molecular imaging technique is positron emission tomography (PET).
